(19) United States
(12) Patent Application Publication
(10) Pub. No.: US 2024/0408200 A1
(43) Pub. Date: Dec. 12, 2024

(54) METHODS OF CELL CULTURE FOR ADOPTIVE CELL THERAPY

(71) Applicants: WILSON WOLF MANUFACTURING, St. Paul, MN (US); BAYLOR COLLEGE OF MEDICINE, Houston, TX (US)

(72) Inventors: Juan F. Vera, Bellaire, TX (US); Cliona M. Rooney, Bellaire, TX (US); Ann M. Leen, Bellaire, TX (US); John R. Wilson, New Brighton, MN (US)

(21) Appl. No.: 18/746,679

(22) Filed: Jun. 18, 2024

Related U.S. Application Data
(63) Continuation of application No. 14/996,447, filed on Jan. 15, 2016, now abandoned.
Application No. 14/996,447 is a division of application No. 13/493,768, filed on Jun. 11, 2012, now abandoned.
Application No. 13/493,768 is a continuation-in-part of application No. 12/963,597, filed on Dec. 8, 2010, now Pat. No. 8,809,050.
(60) Provisional application No. 61/267,761, filed on Dec. 8, 2009.

(51) Int. Cl. A61K 39/00; A61K 9/50; A61K 39/39; A61K 48/00; A61K 51/08; C12N 5/0783; C12N 15/115; C12M 1/04

(52) U.S. Cl. CPC A61K 39/4611; A61K 9/5068; A61K 39/39; A61K 39/4621; A61K 39/4631; A61K 39/46433; A61K 39/46434; A61K 39/464493; A61K 39/464838; A61K 48/0033; A61K 51/08; A61K 2239/26; A61K 2239/48; A61K 2239/58; C12N 5/0636; C12N 5/0638; C12N 15/115; A61K 2039/55516; A61K 2039/55527; A61K 2039/55533; C12M 23/24; C12N 2310/16; C12N 2320/32; C12N 2502/11; C12N 2502/1107

(57) ABSTRACT

Production and use of novel therapeutic cells, called T-Vehicles, in the allogeneic Adoptive Cell Therapy setting allows a wide range of therapeutic benefits to accrue with minimal or no risk of GVHD. T-Vehicles are created from donor T cells that are altered to contain therapeutic attributes that do not include their native antigen receptors and can deliver therapeutic benefits irrelevant of their native antigen specificity. T-Vehicles can possess highly restricted native antigen specificity that renders them unable to recognize antigens present on normal cells and incapable of initiating GVHD, making them ideal transport vehicles to deliver various therapeutic attributes in vivo. In essence, production and use of T-Vehicles is a paradigm shift that opens the door to therapeutic application of T cells in ways not previously contemplated, independent of whether or not there is an HLA match between the donor and the recipient.

### RELATED APPLICATION

The present application is a continuation of U.S. patent application Ser. No. 14/996,447 filed Jan. 15, 2016, which is a divisional of U.S. patent application Ser. No. 13/493,768 filed Jun. 11, 2012, which is a continuation-in-part of U.S. patent Ser. No. 12/963,597, filed Dec. 8, 2010, now U.S. Pat. No. 8,809,050 issued on Aug. 19, 2014, which claims the benefit of U.S. Provisional Application No. 61/267,761, filed Dec. 8, 2009, which are incorporated herein in their entirety by reference.

### FIELD OF THE INVENTION

The present invention relates generally to methods of culturing cells, and more specifically to culturing cells for cell therapy. It further relates to the production of T cells with therapeutic attributes for use in Adoptive Cell Therapy.

### BACKGROUND

Cell culture is major contributor to the cost and complexity of cell therapy. With current methods, the process of culturing the cells is time consuming and expensive. Typically, to produce a large number of cells, an in vitro culture process is undertaken that proceeds in stages. At the earliest stage, the desired cells are a relatively small population within a composition of cells that are placed into cell culture devices. In this stage, the composition of cells typically includes the source of the desired cells (such as peripheral blood mononuclear cells), feeder cells that stimulate growth of the desired cells, and/or antigen presenting. Culture devices and methods that allow the medium that cells reside in to be in a generally undisturbed state are favored since the cells remain relatively undisturbed. Such devices include standard tissue culture plates, flasks, and bags. The culture progresses in stages generally consisting of allowing the cell composition to deplete the medium of growth substrates such as glucose, removing the spent medium, replacing the spent medium with fresh medium, and repeating the process until the desired quantity of desired cells is obtained. Often, the cell composition is moved to other devices to initiate a new stage of production as the desired cell population increases and additional growth surface is needed. However, with conventional methods, the rate of population growth of the desired cells slows as the population of cells upon the growth surface increases. The end result is that it is very time consuming and complicated to produce a sizable population of desired cells.

State of the art production methods for generating T lymphocytes with antigen specificity to Epstein Barr virus (EBV-CTLs) provide an example of production complexity. The conventional method for optimal expansion of EBV-CTLs uses standard 24-well tissue culture plates, each well having 2 cm² of surface area for cells to reside upon and the medium volume restricted to 1 ml/cm² due to gas transfer requirements. The culture process begins by placing a cell composition comprised of PBMC (peripheral blood mononuclear cells) in the presence of an irradiated antigen presenting cell line, which may be a lymphoblastoid cell line (LCL), at a surface density (i.e. cells/cm² of growth surface) ratio of about 40:1 with about 1×10⁶ PBMC/cm² and 2.5×10⁴ irradiated antigen presenting cells/cm². That instigates the population of EBV-CTLs within the cell composition to expand in quantity. After 9 days, EBV-CTLs are selectively expanded again in the presence of irradiated antigen presenting LCL at a new surface density ratio of 4:1, with a minimum surface density of about 2.5×10⁵ EBV-CTL/cm². Medium volume is limited to a maximum ratio of 1 ml/cm² of growth surface area to allow oxygen to reach the cells, which limits growth solutes such as glucose. As a result, the maximum surface density that can be achieved is about 2×10⁶ EBV-CTL/cm². Thus, the maximum weekly cell expansion is about 8-fold (i.e. 2×10⁶ EBV-CTL/cm² divided by 2.5×10⁵ EBV-CTL/cm²) or less. Continued expansion of EBV-CTLs requires weekly transfer of the EBV-CTLs to additional 24-well plates with antigenic re-stimulation, and twice weekly exchanges of medium and growth factors within each well of the 24-well plate. Because conventional methods cause the rate of EBV-CTL population expansion to slow as EBV-CTL surface density approaches the maximum amount possible per well, these manipulations must be repeated over a long production period, often as long as 4-8 weeks, to obtain a sufficient quantity of EBV-CTLs for cell infusions and quality control measures such as sterility, identity, and potency assays.

The culture of EBV-CTLs is but one example of the complex cell production processes inherent to cell therapy. A more practical way of culturing cells for cell therapy that can reduce production time and simultaneously reduce production cost and complexity is needed.

We have created novel methods that increase the population growth rate throughout production, and by so doing, reduce the complexity and time needed to produce cells.

In Adoptive Cell Therapy, T cells with native antigen specificity (i.e. T cells that are directed against a particular peptide derived from a specific target antigen when presented in the context of particular human leukocyte antigen (HLA) allele) have been administered in the autologous and in the partially HLA-matched setting to treat viral infections and target tumors. In all of these cases, the therapeutic benefit derived from the fact that (i) the native T cell receptor recognized the antigen of interest, and (ii) the T cells were administered to a recipient who expressed the HLA allele required to present the targeted peptide.

The first adoptive T cell transfer protocols in the allogeneic hematopoietic stem cell transplant (HSCT) setting were based on the premise that donor peripheral blood contained T cells able to mediate antitumor and/or antiviral activity in the HSCT recipient. Accordingly, donor lymphocyte infusions (DLI) have been extensively used to provide anti-tumor immunity, and to a lesser extent, antiviral immunity. DLIs should contain memory T cells specific for tumors as well as a broad range of viruses, however, while successful for the treatment of a proportion of infections with adenovirus and EBV, the efficacy of this therapy is limited by the low frequency of T cells specific for many common acute viruses (such as rotavirus (RSV) and parainfluenza) and the relatively high frequency of alloreactive T cells. The high ratio of alloreactive T cells to virus-specific T cells is especially problematic in recipients of haploidentical transplants, in whom a higher incidence of graft versus host disease (GVHD) limits the tolerable DLI dose, severely limiting the dose of virus-specific T cells received.

To preserve the benefits and enhance the safety of DLI, strategies for the selective inactivation or removal of recipient-specific alloreactive T cells have been evaluated including Induction of anergy, selective allodepletion to minimize the number of alloreactive T cells administered to a recipient, and use of suicide genes for in vivo destruction of alloreactive T cells that have gone off target.

An alternative strategy to prevent and treat specific viral infections after HSCT is the adoptive transfer of ex vivo-expanded T cells with antiviral activity. The specific expansion of virus-reactive T cells has the advantage of increasing the numbers of virus-specific T cells that can be infused without increasing alloreactive T cells. Infusion of enriched antigen-specific T cells with reactivity against a particular antigen potentially increases therapeutic potency while decreasing undesired off-target effects such as GVHD and this therapeutic modality has proven safe and effective for the treatment of hematological malignancies as well as solid tumors such as melanoma and EBV-associated malignancies such as Hodgkin's lymphoma and nasopharyngeal carcinoma.

Of note, all therapies require using the specificity of the native T cell receptor to recognize the antigen, in the context of a major histocompatibility complex (MHC) molecule through the native T cell receptor (TCR). Therefore, the therapeutic benefit itself depends on the use/administration of HLA-matched or partially matched T cells. For example, to target melanoma cells, one can expand antigen-specific melanoma-directed T cells from donor expressing the HLA haplotype (a) against GP100 (a tumor associated antigen expressed on cancer cells). In this situation the therapeutic benefit is mediated by the specific interaction of the native or natural T cell receptor with the target antigen. However, this interaction can only take place in a compatible HLA setting (i.e. in an autologous setting or in the context of another individual who also expresses HLA). This approach can only be extended for the treatment of multiple patients by the generation of a cell bank containing lines with varying HLA haplotypes and where patients are matched to the most suitable T cell line.

In summary, in all current applications of Adoptive Cell Therapy, the therapeutic attribute of the T cell that provides its therapeutic purpose is the native antigen specificity of the donor T cells. This inherent requires at least a partial HLA match between the donor and the recipient, and in the allogeneic setting creates the potential for off-target effects such as GVHD. Others are proposing elimination of the donor T cells antigen receptors altogether through complex genetic engineering, and re-engineering the T cells to carry chimeric antigen receptors, thereby eliminating all innate recognition capacity of the T cell. However, this further complicates the method of producing T cells, which is already one of the main problems of Adoptive Cell Therapy.

An entirely new approach to Adoptive Cell Therapy that overcomes the existing complications is needed to allow for wider application in mainstream society. We disclose a new paradigm which leaves the antigen specific specificity of the donor T cells intact, but alters the donor T cells with a therapeutic attribute that renders the native antigen specificity of the donor T cells irrelevant to its therapeutic purpose. In essence, this paradigm shift opens the door to therapeutic application of T cells in ways not previously contemplated, independent of whether or not there is an HLA match between the donor and the recipient.

### SUMMARY

It has been discovered that the production of cells for cell therapy can occur in a shorter time period and in a more economical manner than is currently possible by using a staged production process that allows unconventional conditions to periodically be re-established throughout the production process. The unconventional conditions include reduced surface density (i.e. cells/cm²) of desired cells, novel ratios of desired cells to antigen presenting and/or feeder cells, and/or use of growth surfaces comprised of gas permeable material with increased medium volume to surface area ratios.

Embodiments of this invention relate to improved methods of culturing cells for cell therapy applications. They include methods that reduce the time, cost, and complexity needed to generate a desired number of desired cells by use of various novel methods that allow the desired cell population to maintain a higher growth rate throughout the production process relative to conventional methods.

One aspect of the present invention relies on conducting the culture process in stages and establishing conditions at the onset of one or more stages that allow the growth rate of the desired cell population to exceed what is currently possible. At least one stage of culture, and preferably nearly all, establish initial conditions that include the desired cells resting either on non-gas permeable or gas permeable growth surfaces at unconventionally low surface density and at an unconventional ratio of antigen presenting cells (and/or feeder cells) per desired cell. By using the novel embodiments of this aspect of the invention, the desired cell population can experience more doublings in a shorter period of time than allowed by conventional methods, thereby reducing the duration of production.

Another aspect of the present invention relies on conducting the culture process in stages and establishing conditions at the onset of one or more stages such that the growth rate of the desired cell population exceeds what is currently possible. At least one stage of culture, and preferably nearly all, establish conditions that include the desired cells resting on a growth surface comprised of gas permeable material at unconventionally high medium volume to growth surface area ratios. By using the novel embodiments of this aspect of the invention, the desired cell population can experience more doublings in a shorter period of time than is allowed by conventional methods, thereby reducing the duration of production.

Another aspect of the present invention relies on conducting the culture process in stages and establishing conditions of each stage such that the growth rate of the desired cell population exceeds what is currently possible. At least one stage of culture, and preferably nearly all, establish initial conditions that include the desired cells resting on growth surfaces comprised of gas permeable material at unconventionally low surface density (i.e. cells/cm²) with an unconventional ratio of antigen presenting cells (and/or feeder cells) per desired cell and in the presence of unconventionally high medium volume to growth surface area ratios. By using the novel embodiments of this aspect of the invention, the desired cell population can experience more doublings in a shorter period of time than conventional methods allow, thereby reducing the duration of production.

In embodiments of the present invention, allogeneic T-Vehicles are created with therapeutic attributes that have a therapeutic purpose that will benefit recipients while not exposing the recipient to graft-versus-host-disease (GVHD).

In one embodiment of the present invention, a therapeutic treatment is undertaken by obtaining T-Vehicles that are created by a process comprising stimulating donor PBMCs or donor cord blood with an antigen in order to activate the growth of T cells that have native antigen specificity to the antigen(s). Doing so produces an antigen-specific T cell population that is comprised of native antigen receptors that have antigen specificity to the antigen(s) that were used to stimulate their growth. The antigen-specific T cell population is altered to include at least one therapeutic attribute which does not include the native antigen receptors and has a therapeutic purpose that is independent of the antigen specificity of the native antigen receptors, thereby creating a population of T-Vehicles. The T-Vehicles are then delivered to a recipient that can derive therapeutic benefit from the T-Vehicles, independent of whether or not the cells of the recipient present antigen recognized by the native antigen receptor(s) of the T-Vehicles and/or wherein the cells of the recipient do not present antigen recognized by the native antigen receptor(s) of the T-Vehicles

In another embodiment of the present invention, a therapeutic treatment is undertaken by obtaining T-Vehicles that are created by a process comprising stimulating donor PBMCs or donor cord blood with an antigen in order to activate the growth of T cells that have native antigen specificity to the antigen(s). Doing so produces an antigen-specific T cell population that is comprised of native antigen receptors that have antigen specificity to the antigen(s) that were used to stimulate their growth. The antigen-specific T cell population is altered to include at least one therapeutic attribute which does not include the native antigen receptors and has a therapeutic purpose that is independent of the antigen specificity of the native antigen receptors, thereby creating a population of T-Vehicles. The T-Vehicles are then delivered to a recipient that can derive therapeutic benefit from the T-Vehicles and does not have an HLA match to the T-Vehicles.

In various embodiments of the present invention, T-Vehicles are altered to become loaded with recombinant proteins administered as an adjuvant with immunotherapies, altered with the therapeutic attribute of chemotherapeutic agents for the targeted treatment of cancer, altered with the therapeutic attribute of antimicrobial agents, altered with the therapeutic attribute of expressing transgenic molecules that confer the cells with tumor specificity, altered with the therapeutic attribute of being loaded or engineered with recombinant proteins for the treatment of autoimmune diseases, altered to express suicide genes, and/or are altered with the therapeutic attribute of loaded and/or engineered to in-vivo imaging.

In another embodiment of the present invention, a method of producing antigen specific T cells with desired antigen recognition is attained by placing PBMCs or cord blood into a cell culture device, adding more than one antigen into the cell culture device in order to activate the growth of more than one population of antigen specific T cells, each population capable of recognizing one of the antigens, allowing a period of time for the antigen specific T cells to initiate population expansion, assessing the culture to determine the presence and/or quantity of at least one population of antigen specific T cells, determining which of the populations of T cells is suitable for continued proliferation, and re-stimulating the culture only with antigens recognized by the suitable populations of T cells.

In another embodiment of the present invention, a method of producing antigen specific T cells with desired antigen recognition is attained by placing PBMCs or cord blood into a cell culture device, initially adding more than one antigen into the cell culture device in order to activate the growth of more than one population of antigen specific T cells, each population capable of recognizing one of the antigens, allowing a period of time for the antigen specific T cells to initiate population expansion, separating the culture into more than one device, adding only one of the initial antigens into each device, and determining which of the devices contains a population of antigen specific T cells suitable for continued proliferation, and terminating the culture in devices that do not contain a population of antigen specific T cells suitable for continued proliferation.

In various embodiments of the present invention, donor T cells are produced with native antigen specificity that only allows them to recognize a single eptitope of antigens that are not present on normal human cells and not present on normal mammalian cells.

### BRIEF DESCRIPTION OF THE DRAWINGS

The invention may be more completely understood in consideration of the following detailed description of various embodiments of the invention in connection with the accompanying drawings, in which:

FIG. 1A shows the population of antigen-specific T cells in Example 1 undergoes at least 7 cell doublings after the initial stimulation over the first 7 days.

FIG. 1B shows data demonstrating the magnitude of expansion of a T cell population within a cell composition over time as determined by tetramer analysis for Example 1.

FIG. 1C the rate of population growth of antigen-specific T cells diminishes over a 23 day period in Example 1.

FIG. 2 shows a table that illustrates the discrepancy between the potential expansion and observed fold expansion of antigen-specific T cells in Example 1.

FIG. 3A shows the presence of antigen-specific T cells following stimulations in Example 2.

FIG. 3B shows the expansion of a population of antigen-specific T cells as surface densities diminish from 1×10⁶/cm² to 3.1×10⁴/cm² while maintaining an antigen-specific T cell to antigen presenting cell ratio of 4:1 in Example 2.

FIG. 3C shows the expansion of a population of antigen-specific T cells as surface densities diminish from 1×10⁶/cm² to 3.1×10⁴/cm² while in the presence of a fixed number of antigen presenting cells in Example 2.

FIG. 4 shows an example of results obtained when continuing the work described in FIG. 3, which further demonstrated that when desired cells need the support of other cells, unconventionally low desired cell surface density can initiate population expansion so long as desired cells are in the presence of an adequate supply of feeder and/or antigen presenting cells.

FIG. 5 shows a histogram demonstrating the ability to repeat the magnitude of the population expansion of desired cells by initiating culture at three differing cell surface densities (CTL/cm²).

FIG. 6 shows a cross-sectional view of a gas permeable test fixture used to generate data.

FIG. 7A shows the growth curves of antigen-specific T cells produced in accordance with the present invention in comparison to conventional methods as undertaken in Example 5.

FIG. 7B shows that for Example 5, cell viability was significantly higher in antigen-specific T cells produced in accordance with the present invention in comparison to conventional methods as determined by flow cytometric forward vs. side scatter analysis.

FIG. 7C shows that for Example 5, cell viability was significantly higher in antigen-specific t cells produced in accordance with the present invention in comparison to conventional methods as determined by Annexin-PI 7AAD.

FIG. 7D showed that for Example 5, the superior growth of cells produced in the novel methods of the present invention exhibited the same cell specific growth rate as cell cultured using conventional methods as determined by daily flow cytometric analysis of CFSE labeled cells, confirming that the increased rate of cell expansion resulted from decreased cell death.

FIG. 8A shows how EVB-CTLs were able to expand beyond what was possible in conventional methods without need to exchange medium.

FIG. 8B shows how the culture condition of Example 6 did not modify the final cell product as evaluated by Q-PCR for EBER.

FIG. 8C shows how the culture condition of Example 6 did not modify the final cell product as evaluated by Q-PCR for B cell marker CD20.

FIG. 9 shows an illustrative example in which we experimentally demonstrated that a very low cumulative surface density of desired cells and antigen presenting cells (in this case AL-CTLs and LCLs cells combining to create a cell composition with a surface density of 30,000 cells/cm²) was unable to initiate outgrowth of the AL-CTL population.

FIG. 10A presents data of Example 8 that show how two novel methods of culturing cells produce more cells over a 23 day period than a conventional method.

FIG. 10B shows a photograph of cells cultured in a test fixture in Example 8.

FIG. 10C shows that in Example 8, the two novel methods of culture and the conventional method all produce cells with the same phenotype.

FIG. 10D shows that for Example 8, a representative culture in which T cells stimulated with EBV peptide epitopes from LMP1, LMP2, BZLF1 and EBNA1 of EBV and stained with HLA-A2-LMP2 peptide pentamers staining showed similar frequencies of peptide-specific T cells.

FIG. 10E shows that for the novel methods and the conventional method of Example 8, cells maintained their cytolytic activity and specificity and killed autologous EBV-LCL, with low killing of the HLA mismatched EBV-LCL as evaluated by ⁵¹Cr release assays.

FIG. 11 shows a graphical representation of expansion of a desired cell population on a growth surface under the conventional scenario as compared to population expansion of the desired cell type using one aspect of the present invention.

FIG. 12 shows an example of the advantages that can be obtained by utilizing a growth surface comprised of gas permeable material and an unconventionally high medium volume to growth surface area ratio beyond 1 or 2 ml/cm².

FIG. 13 shows a graphical representation of a novel method of expansion of a desired cell population on a growth surface under the conventional scenario as compared to population expansion of the desired cell type under one embodiment of the present invention in which the cell surface density at the completion of is much greater than conventional surface density.

FIG. 14 shows another novel method of cell production that provides yet further advantages over conventional methods.

FIG. 15 shows a comparison of each production method depicted in FIG. 14 to demonstrate the power of the novel method and why it is useful to adjust the production protocol at various stages to fully capture the efficiency.

FIG. 16 shows an example of how one could adjust the production protocol in the novel method to gain efficiency as production progresses.

FIG. 17 shows test results demonstrating T-Vehicles are unable to recognize cells from mismatched allogeneic donors.

FIG. 18 shows test results indicating donor T cells can be altered to create T-Vehicles with the therapeutic attribute of CD34Δ-IL7 cytokine expression, as determined by flow analysis.

FIG. 19A shows test results indicating systemic delivery of IL7 cytokine results in more cytokine being detected on the kidney of mice than at their tumor site.

FIG. 19B shows test results indicating T-Vehicle delivery of IL7 cytokine results in greater cytokine concentration at the mice tumor sites, when compared with other organs, and shows how cytokine production was sustained at the tumor for at least 2 weeks after the administration of the T-vehicles.

FIG. 20 shows test results indicating donor T cells can be altered to create T-Vehicles with the therapeutic attribute of CAR-PSCA, as determined by flow analysis.

FIG. 21 shows test results indicating T-Vehicles with the therapeutic attribute of CAR-PSCA are able to eradicate tumor cells.

FIG. 22A shows T-Vehicles with receptors capable of binding IL4 are in proximity of tumor cells expressing IL4 cytokine.

FIG. 22B shows how T-Vehicles can bind IL4 cytokines, and the quantity of IL4 cytokines protecting the tumor cells can be greatly reduced.

FIG. 23 shows test results demonstrating T-Vehicles, having a therapeutic attribute of expressing extra-cellular recombinant cytokine receptors IL4R/7, are able to deplete IL4 cytokine.

FIG. 24A shows how T-Vehicles loaded with chemotherapeutic agent will migrate towards the site of inflammation.

FIG. 24B shows how the Recipient immune system will target the T-Vehicles, which are located at the site of the Tumor cells.

FIG. 24C shows how, under attack by the Recipient immune system, T-Vehicles will release their payload, in this case a chemotherapeutic agent, at the site of the Tumor cells.

### DETAILED DESCRIPTION

### Definitions

Antigen presenting cells (APC): Cells that act to trigger the desired cells to respond to a particular antigen.
CTL: Cytotoxic T cell
Desired cells: The specific type of cell that that the production process aims to expand in quantity. Generally the desired cells are non-adherent and examples includie regulatory T cells (Treg), natural killer cells (NK), tumor infiltrating lymphocytes (TIL), primary T lymphocytes and a wide variety of antigen specific cells, and many others (all of which can also be genetically modified to improve their function, in-vivo persistence or safety). Cells required for clinical use can be expanded with feeder cells and/or antigen presenting cells that can include PBMC, PHA blast, OKT3 T, B blast, LCLs and K562, (natural or genetically modified to express and antigen and/or epitope as well as co-stimulatory molecules such as 41BBL, OX40, CD80, CD86, HLA, and many others) which may or may not be pulsed with peptide or other relevant antigens.

### EBV: Epstein Barr Virus

EBV-CTL: A T cell that specifically recognized EBV-infected cells or cells expressing or presenting EBV-derived peptides through its T cell surface receptor.
EBV-LCL: Epstein Barr virus transformed B lymphoblastoid cell line.
Feeder cells: Cells that act to cause the desired cells to expand in quantity. Antigen presenting cells can also act as feeder cells in some circumstances.
Growth surface: The area within a culture device upon which cells rest.
PBMCs: Peripheral Blood Mononuclear Cells derived from peripheral blood, which are a source of some of the desired cells and which can act as feeder cells.
Responder (R): A cell that will react to a stimulator cell.
Static cell culture: A method of culturing cells in medium that is not stirred or mixed except for occasions when the culture device is moved from location to location for routine handling and/or when cells are periodically fed with fresh medium and the like. In general, medium in static culture is typically in a quiescent state. This invention is directed to static cell culture methods.
Stimulated: The effect that antigen presenting and/or feeder cells have on the desired cells.
Stimulator (S): A cell that will influence a responder cell.
Surface density: The quantity of cells per unit area of the surface within the device upon which the cells rest.

In attempting to find novel methods to simplify the production of a desired population of cells for adoptive T cell therapy, a series of experiments were conducted that have that opened the door to more efficient culture of cells for cell therapy applications. Numerous illustrative examples and various aspects of the present invention are described to indicate how the ability to reduce production time and complexity relative to conventional methods can be achieved.

### Example 1

### Demonstration of Limitations of Conventional Methods

The data of this example demonstrate the limits of conventional culture methods for the production of EBV-CTL in standard 24 well tissue culture plates (i.e. 2 cm² surface area per well) using a medium volume of 2 ml per well (i.e. medium height at 1.0 cm and a medium volume to surface area ratio of 1 ml/cm²).

Stage 1 of culture, day 0: The expansion of an EBV-CTL population was initiated by culturing a cell composition of PBMCs from normal donors (about 1×10⁶ cells/ml) with antigen presenting gamma-irradiated (40 Gy) autologous EBV-LCLs at a 40:1 ratio (PBMC:LCLs) and a medium volume to growth surface ratio of 1 ml/cm² thereby establishing a cell composition surface density of about 1×10⁶ cells/cm² in RPMI 1640 supplemented with 45% Click medium (Irvine Scientific, Santa Ana, CA), with 2 mM GlutaMAX-I, and 10% FBS.
Stage 2 of culture, day 9-16: On day 9, EBV-CTLs were harvested from the cell composition created in Stage 1, resuspended in fresh medium at a surface density of 0.5×10⁶ EBV-CTL/cm² and re-stimulated with irradiated autologous EBV-LCLs at a ratio 4:1 CTL:LCL (surface density 0.5×10⁶ CTL/cm²:1.25×10⁵ LCL/cm²). On day 13, 1 ml of the 2 ml medium volume in each well of the 24-well plates was removed and replaced with 1 ml of fresh medium containing recombinant human IL-2 (IL-2) (50 U/mL) (Proleukin; Chiron, Emeryville, CA)
Stage 3 of culture, day 17-23: The conditions of Stage 2 were repeated with twice weekly addition of IL-2 and the culture was terminated on day 23. Although the culture was terminated, it could have been continued with additional culture stages that mimicked that of stages 2 and 3.
Cell lines and tumor cells for use as target cells in Cytotoxicity assays: BJAB (a B cell lymphoma) and K562 (a chronic erythroid leukemia) were obtained from the American Type Culture Collection (ATCC, Rockville, MD, USA). All cells were maintained in culture with RPMI 1640 medium (GIBCO-BRL, Gaithersburg, MD) containing 10% heat-inactivated fetal calf serum (FCS), 2 mM L-glutamine, 25 IU/mL penicillin, and 25 mg/mL streptomycin (all from BioWhittaker, Walkersville, MD). Cells were maintained in a humidified atmosphere containing 5% CO₂ at 37° C.

### Immunophenotyping:

Cell surface: Cells were stained with Phycoerythrin (PE), fluorescein isothiocyanate (FITC), periodin chlorophyll protein (PerCP) and allophycocyanin (APC)-conjugated monoclonal antibodies (MAbs) to CD3, CD4, CD8, CD56, CD16, CD62L, CD45RO, CD45RA, CD27, CD28, CD25, CD44 from Becton-Dickinson (Mountain View, CA, USA). PE-conjugated tetramers (Baylor College of Medicine) and APC-conjugated pentamers (Proimmune Ltd, Oxford, UK), were used to quantify EBV-CTL precursor frequencies. For cell surface and pentamer staining 10,000 and 100,000 live events, respectively, were acquired on a FACSCalibur flow cytometer and the data analyzed using Cell Quest software (Becton Dickinson).
CFSE labeling to measure cell division: To assess the doubling rate of 2×10⁷ PBMC or EBV-specific CTLs (EBV-CTLs) were washed twice and resuspended in 850 μl 1× phosphate-buffered saline (PBS) containing 0.1% Fetal Bovine Serum (FBS) (Sigma-Aldrich). Prior to staining, an aliquot of carboxy-fluorescein diacetate, succinimidyl ester (CFSE) (10 mM in dimethyl sulfoxide) (Celltrace™ CFSE cell proliferation kit (C34554) Invitrogen) was thawed, diluted 1:1000 with 1× PBS and 150ul of the dilution was added to the cell suspension (labeling concentration was 1 μM). Cells were incubated with CFSE for 10 minutes at room temperature. Subsequently 1 ml FBS was added to the cell suspension followed by a 10 minute incubation at 37° C. Afterwards cells were washed twice with 1× PBS, counted, and stimulated with antigen as described.
AnnexinV-7-AAD staining: To determine the percentage of apoptotic and necrotic cells in our cultures we performed Annexin-7-AAD staining as per manufacturers' instructions (BD Pharmingen™ #559763, San Diego, CA). Briefly, EBV-CTL from the 24-well plates or the G-Rex were washed with cold PBS, resuspended in 1X Binding Buffer at a concentration of 1×10⁶ cells/ml, stained with Annexin V-PE and 7-AAD for 15 minutes at RT (25° C.) in the dark. Following the incubation the cells were analyzed immediately by flow cytometry.
Chromium release assay: We evaluated the cytotoxic activity of EBV-CTLs in standard 4-hour ⁵¹Cr release assay, as previously described. As desired cells we used autologous and HLA class I and II mismatched EBV-transformed lymphoblastoid cell line (EBV-LCL) to measure MHC restricted and unrestricted killing, as well as the K562 cell line to measure natural killer activity. Chromium-labeled desired cells incubated in medium alone or in 1% Triton X-100 were used to determine spontaneous and maximum ⁵¹Cr release, respectively. The mean percentage of specific lysis of triplicate wells was calculated as follows: [(test counts−spontaneous counts)/(maximum counts−spontaneous counts)]×100.
Enzyme-Linked Immunospot (ELIspot) assay: ELIspot analysis was used to quantify the frequency and function of T cells that secreted IFNγ in response antigen stimulation. CTL lines expanded in 24 well plates or in the G-Rex were stimulated with irradiated LCL (40Gy) or LMP1, LMP2, BZLF1 and EBNA1 pepmixes (diluted to 1 μg/ml) (JPT Technologies GmbH, Berlin, Germany), or EBV peptides HLA-A2 GLCTLVAML=GLC, HLA-A2 CLGGLLTMV=CLG, HLA-A2-FLYALALLL=FLY, and HLA-A29 ILLARLFLY=ILL (Genemed Synthesis, Inc. San Antonio, Texas), diluted to a final concentration of 2 μM, and CTLs alone served as a negative controls. CTLs were resuspended at 1×10⁶/ml in ELIspot medium [(RPMI 1640 (Hyclone, Logan, UT) supplemented with 5% Human Serum (Valley Biomedical, Inc., Winchester, Virginia) and 2-mM L-glutamine (GlutaMAX-I, Invitrogen, Carlsbad, CA)]. Ninety-six-well filtration plates (MultiScreen, #MAHAS4510, Millipore, Bedford, MA) were coated with 10 μg/mL anti-IFN-γ antibody (Catcher-mAB91-DIK, Mabtech, Cincinnati, OH) overnight at 4° C., then washed and blocked with ELIspot medium for 1 hour at 37° C. Responder and stimulator cells were incubated on the plates for 20 hours, then the plates were washed and incubated with the secondary biotin conjugated anti-IFN-γ monoclonal antibody (Detector-mAB (7-B6-1-Biotin), Mabtech) followed by incubation with Avidin: biotinylated horseradish peroxidase complex (Vectastain Elite ABC Kit (Standard), #PK6100, Vector Laboratories, Burlingame, CA) and then developed with AEC substrate (Sigma, St. Louis, MO). Each culture condition was run in triplicate. Plates were sent for evaluation to Zellnet Consulting, New York, NY. Spot-forming units (SFC) and input cell numbers were plotted.
Statistical analysis: In vitro data are presented as mean±1 SD. Student's t test was used to determine the statistical significance of differences between samples, and P<0.05 was accepted as indicating a significant difference.

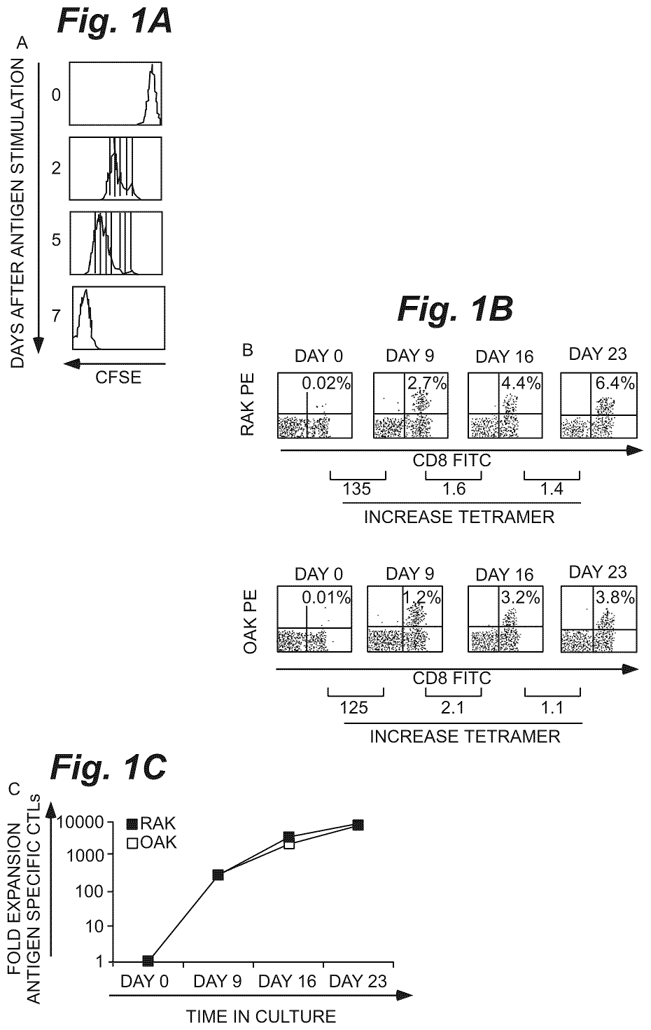

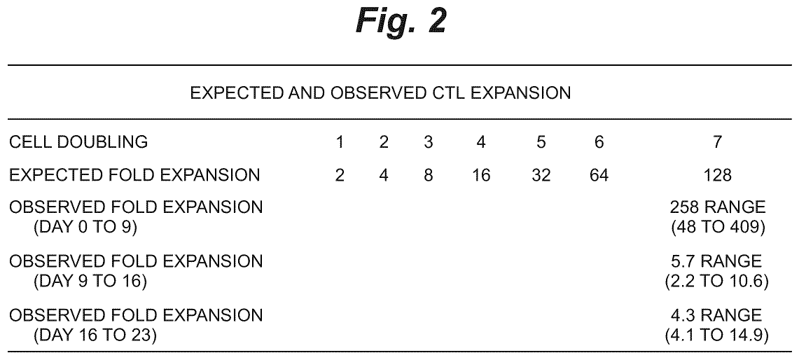

Under these culture conditions, the population of antigen-specific T cells undergoes at least 7 cell doublings after the initial stimulation over the first 7 days, as shown in FIG. 1A. Thus we expect a weekly T cell expansion of 128-fold (as measured by the frequency of antigen-specific T cells times the total number of cells in the cell composition). The frequency of tetramer positive cells after the first, second, and third stimulations is shown in FIG. 1B. On day 0 the frequency of T cells reactive against two EBV tetramers, RAK and QAK was 0.02% and 0.01%, respectively. After a single stimulation on day 0, by day 9 the frequency of tetramer-positive T cells in the cell composition had increased from 0.02% and 0.01% to 2.7% and 1.25%, respectively. Thus, a 135-fold and 125-fold increase in the percentage of antigen-specific tetramer positive T cells residing within the cell composition was attained as measured by RAK and QAK. Also, after a single stimulation on stage 1 of culture, day 0, a 1.1 fold increase in the surface density of cells in the cell composition (data not shown) was observed by day 9 (approximately 1.1×10⁶ cells/cm² were present). Since the majority of cells within the PBMC composition are not specific for the stimulating antigens, little overall increase in total cell number is observed, but the fold expansion of the antigen-specific cell population within the composition was around 280 during the first stage of culture, as shown in FIG. 1C. Unfortunately, although the number of cell doublings was the same during the second and third stages of culture as measured by CSFE, this rate of antigen-specific T cell expansion was not sustained during the 2^{nd} or the 3^{rd} stages of culture, being only 5.7 in stage two and 4.3 in stage three. FIG. 2 shows a table that illustrates the discrepancy between the potential expansion and observed fold expansion of antigen-specific T cells (n=3).

Example 1 demonstrates that the amount of time it takes to produce the desired cells is typically delayed after roughly the first week of production since the rate of population expansion of the desired cells decreases in subsequent stages of culture.

### Example 2

Reducing the amount of time needed to increase the desired cell population can be achieved by reducing the cell surface density of the desired cell population as the onset of any given stage or stages of culture.

We hypothesized that the decreased rate of expansion of the desired cell population following the second T cell stimulation compared to the first stimulation was due to limiting cell culture conditions that resulted in activation induced cell death (AICD). For example, referring to

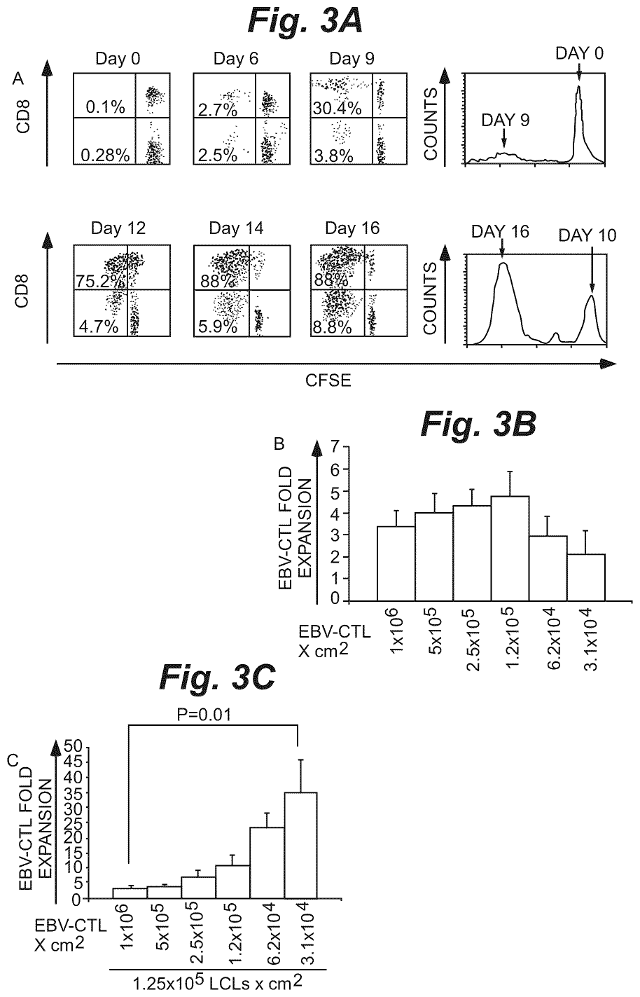

FIG. 3A, at the first stimulation, the EBV antigen-specific T cell component of PBMCs represents, at most, 2% of the population and so the antigen-specific responder T cell seeding density is less than 2×10⁴ per cm², with the remaining PBMC acting as non-proliferating feeder cells (seen as the CFSE positive cells in FIG. 3A) that sustain optimal cell-to-cell contact allowing proliferation of the antigen-specific CTLs. By contrast, at the second stimulation on day 9, the majority of T cells are antigen-specific, and although the total cell density of the composition is about the same, the proliferating cell density is 50 to 100 fold higher. As a consequence, on re-stimulation the majority of cells proliferate and may therefore rapidly consume and exhaust their nutrients and O₂ supply.

To determine whether limiting culture conditions were responsible for sub-optimal T cell growth rates, we measured the expansion of activated T cells plated at lower cell densities. Methods were as previously described in Example 1.

We seeded activated EBV-specific T cells in wells of standard 24-well plates, each well having 2 cm² of growth surface area, at doubling dilutions to create diminishing surface densities ranging from 1×10⁶/cm² to 3.1×10⁴/cm² while maintaining a responder cell to stimulatory cell ratio (R:S) of 4:1 as shown in FIG. 3B. The maximum CTL expansion (4.7±1.1 fold) was achieved with a starting CTL surface density of 1.25×10⁵ per cm², but further dilution decreased the rate of expansion as shown in FIG. 3B. We speculated that this limiting dilution effect was possibly due to lack of cell-to-cell contact, and therefore we cultured doubling dilutions of EBV-CTL from surface densities of 1×10⁶ to 3.1×10⁴ with a fixed number of feeder cells (EBV-LCL plated at a surface density of 1.25×10⁵/cm²) and assessed cell expansion over a 7 day period. We observed a dramatic increase in CTL expansion from merely 2.9±0.8 fold with EBV-CTL at a surface density of 1×10⁶/cm² all the way to a 34.7±11 fold expansion with EBV-CTL at a surface density of 3.1×10⁴/cm², as presented in FIG. 3C. Importantly, this modification of the culture conditions did not change the function or antigen specificity of the cells (data not shown). A population of activated antigen-specific T cells is therefore capable of greater expansion than conventional culture methods allow. Of note, the maximum surface density achieved after stimulation (1.7 to 2.5×10⁶/cm²) was the same regardless of the starting surface density.

Thus, conventional culture conditions were limiting, indicating the medium volume to growth surface area ratio needs to increase beyond the conventional 1 ml/cm² to allow the desired cell population to move beyond the surface density limits of conventional methods. Additionally, improved expansion of antigen-specific CTL to about 34-fold can be obtained by reducing the surface density of the desired cell population below conventional methods at the onset of any stage of culture. This has substantial ramifications in cell therapy, where the quantity of cells at the onset of production is often quite limited. For example, by distributing the in limited amount of desired cells onto increased surface area at lowered surface density, a greater population of desired cells can be attained in a shorter period of time as the rate of population growth increases dramatically relative to conventional surface density.

### Example 3

A minimum surface density of a cell population that includes the desired cells and/or antigen presenting cells can allow outgrowth of a desired cell population that is seeded at very low surface density.

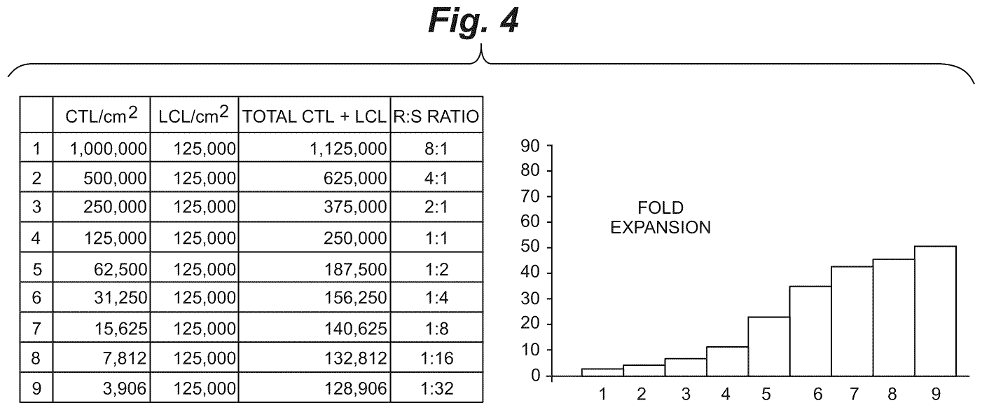

FIG. 4 shows an example of results we obtained when continuing the work described in FIG. 3, which further demonstrated that when desired cells need the support of other cells, unconventionally low desired cell surface density can initiate population expansion so long as desired cells are in the presence of an adequate supply of feeder and/or antigen presenting cells. In these experiments, we continue to demonstrate how a total cell composition with a surface density and R:S ratio of between about 1.0×10⁶ desired cells/cm² at an R:S ratio of 8 to 1 and merely about 3900 desired cells/cm² at an R:S ratio of 1 to 32 could allow desired cells to be greatly expanded to over 50 fold times the starting surface density, at which point we discontinued testing.

### Example 4

The ability to allow a production process to repeat in stages by initiating a stage with an unconventionally low desired cell surface density, allowing population expansion, terminating the stage and repeating conditions was demonstrated to deliver repeatable outcomes.

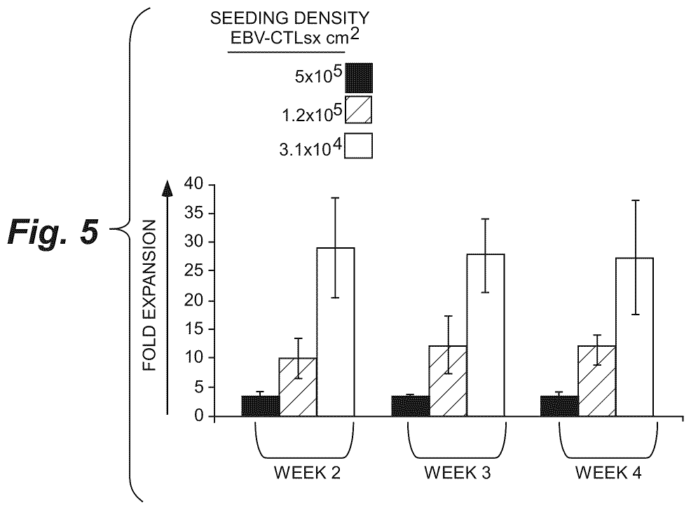

We continued the assessments described in Example 3 at three of the desired cell surface densities (CTL/cm²) as shown in FIG. 5. Each specific seeding density was able to consistently attain the same fold expansion. The implications will be described in more detail further on as they relate to the ability to dramatically reduce the production time for a desired cell population.

### Example 5

Culturing desired cells on a growth surface that is comprised of gas permeable material while simultaneously increasing the medium volume to growth surface area ratio increases the number of times a desired cell population can double in a given stage of culture relative to conventional methods and increases the surface density that is attainable.

Cell lines and tumor cells, immunophenotyping, CFSE labeling, AnnexinV-7-AAD staining, chromium release assay, Enzyme-Linked Immunospot (ELIspot) assay, retrovirus production and transduction of T-lymphocytes, and statistical analysis were as described in Example 1.

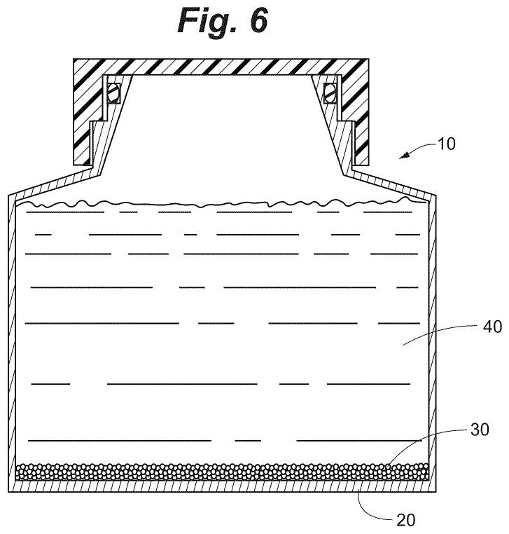

Test fixtures (hereinafter generically referred to as “G-Rex”) were constructed as shown in FIG. 6. Bottom 20 of each G-Rex 10 was comprised of gas permeable silicone membrane, approximately 0.005 to 0.007 inches thick. Co-pending U.S. Patent Publication US 2005/0106717 A1 (hereinafter referred to as Wilson '717) is among many other sources of information relating to the use of alternative gas permeable materials and can be used to educate skilled artisans about gas permeable culture device shapes, features, and other useful characteristics that are beneficial to many of the embodiments of this invention. In this Example 3, G-Rex (referred to as “G-Rex40”) had a growth surface area of 10 cm², upon which a cell composition (shown as item 30) rested, the characteristics of the cell composition varied throughout the experiment as described within. Medium volume (shown as item 40) unless otherwise indicated was 30 mL, creating a medium volume to growth surface area ratio of 3 ml/cm².

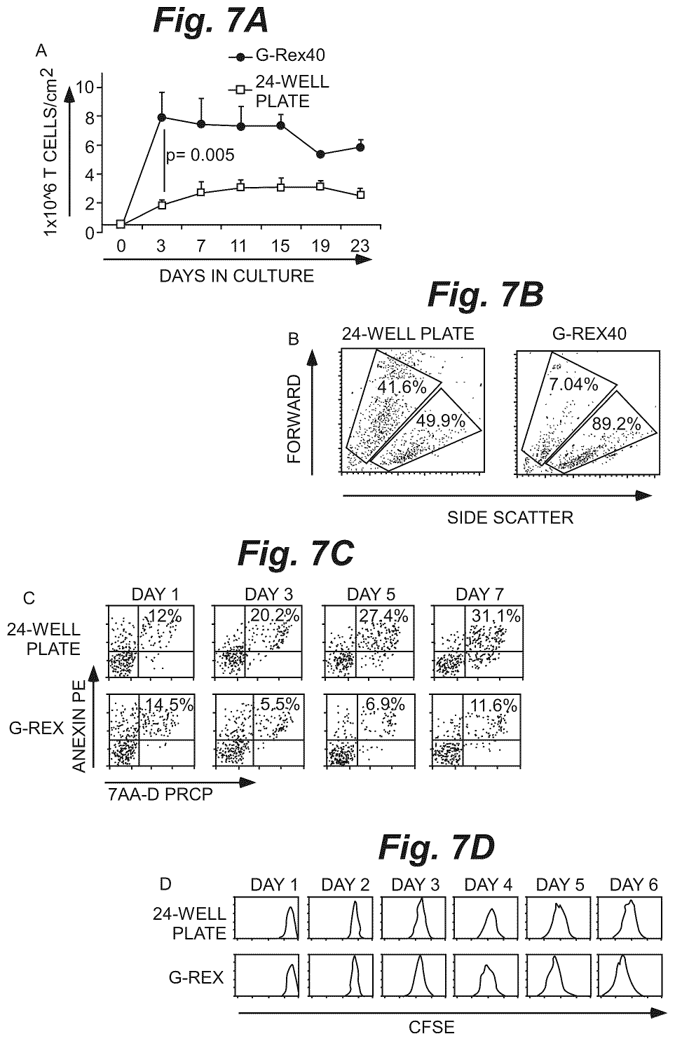

Activated EBV-specific CTL and irradiated autologous EBV-LCLs at the conventional 4:1 ratio of CTL: LCL were cultured in G-Rex40 devices. EBV-CTLs were seeded at a surface density of 5×10⁵ cells/cm² in the G-Rex40 and the rate of EBV-CTL population expansion was compared with EBV-CTL seeded at the same surface density in a standard 24-well plate with a medium volume to growth surface area of 1 ml/cm². After 3 days, as shown in FIG. 7A (p=0.005), the EBV-CTLs in the G-Rex40 had increased from 5×10⁵/cm² to a median of 7.9×10⁶/cm² (range 5.7 to 8.1×10⁶/cm²) without any medium exchange. In contrast, EBV-CTLs cultured for 3 days in conventional 24-well plates only increased from a surface density of 5×10⁵/cm² to a median of 1.8×10⁶/cm² (range 1.7 to 2.5×10⁶/cm²) by day 3. In the G-Rex40, surface density could be further increased by replenishing medium whereas cell surface density could not be increased by replenishing medium or IL2 in the 24-well plate. For example, EBV-CTL surface density further increased in the G-Rex40 to 9.5×10⁶ cells/cm² (range 8.5×10⁶ to 11.0 ×10⁶/cm²) after replenishing the medium and IL-2 on day 7 (data not shown).

To understand the mechanism behind the superior cell expansion in the G-Rex device, we assessed the viability of OKT3-stimulated peripheral blood T cells using flow cytometric forward vs. side scatter analysis on day 5 of culture. EBV-CTLs could not be assessed in this assay due to the presence of residual irradiated EBV-LCL in the cultures, which would interfere with the analysis. As shown in FIG. 7B, cell viability was significantly higher in the G-Rex40 cultures was significantly higher (89.2% viability in the G-Rex40 vs. 49.9% viability in the 24-well plate). We then analyzed the cultures each day for 7 days using Annexin-PI 7AAD to distinguish between live and apoptotic/necrotic cells, and observed consistently lower viability in T cells expanded in 24 well plates compared to those in the G-Rex, as shown in FIG. 7C. These data indicate the cumulative improved survival of proliferating cells contributed to the increased cell numbers in the G-Rex devices compared to the 24-well plates.

To determine if there was also a contribution from an increased number of cell divisions in the G-Rex versus the 24-well plates, T cells were labeled with CFSE on day 0 and divided between a G-Rex40 device with a 40 ml medium volume and a 24 well plate with each well at a 2 ml medium volume. Daily flow cytometric analysis demonstrated no differences in the number of cell divisions from day 1 to day 3. From day 3 onwards, however, the population of desired cells cultured in the G-Rex40 continued to increase at a rate that exceeded the diminishing rate of the 2ml wells, indicating that the culture conditions had become limiting as shown in FIG. 7D. Thus, the large population of desired cells in the G-Rex40 test fixtures resulted from a combination of decreased cell death and sustained proliferation relative to conventional methods.

### Example 6

By use of unconventionally high ratios of medium volume to growth surface area and use of growth surfaces comprised of gas permeable material, the need to feed culture during production can be reduced while simultaneously obtaining unconventionally high desired cell surface density.

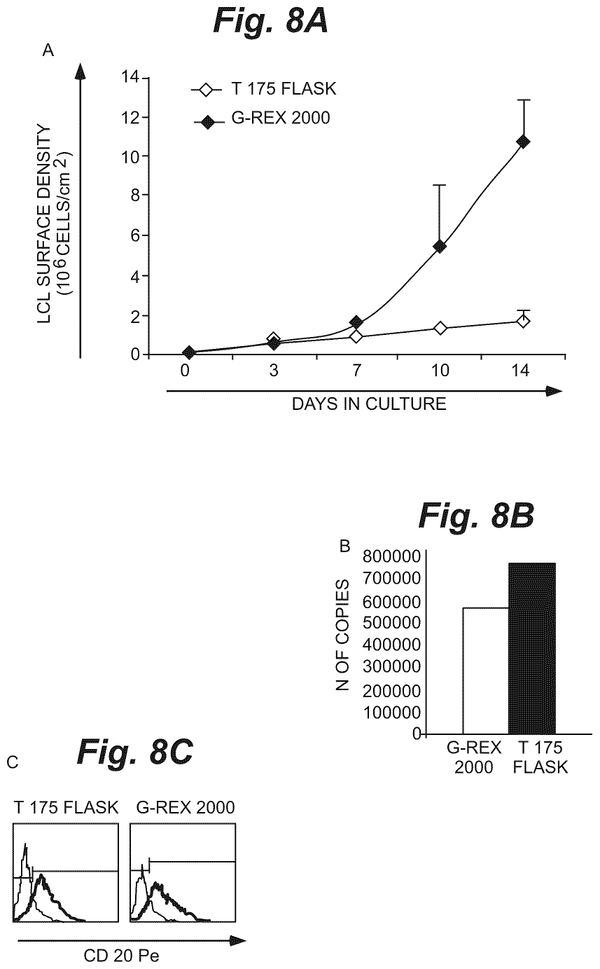

This was demonstrated through use of G-Rex test fixtures for the initiation and expansion of EBV: LCLs. For purposes of this example, G-Rex2000 refers to device as described in FIG. 8, the exception being the bottom is comprised of a 100 cm² growth surface area and a 2000 ml medium volume capacity is available. EBV-LCLs were cultured in and expand in the G-Rex2000without changing the cell phenotype. EBV-LCL were plated into a G-Rex2000 at a surface density of 1×10⁵ cells/cm² along with 1000 ml of complete RPMI medium to create a medium volume to surface area ratio of 10 ml/cm². For comparison, EBV-LCL were plated into a T175 flask at a surface density of 5×10⁵ cells/cm² along with 30 ml of complete RPMI medium to create a medium volume to surface area ratio of about 0.18 ml/cm². As presented in FIG. 8A, the EBV-LCL cultured in G-Rex2000 expanded more than those in the T175 flask without requiring any manipulation or media change. This culture condition did not modify the final cell product as evaluated by Q-PCR for EBER and B cell marker CD20 as presented in FIG. 8B and FIG. 8C.

### Example 7

When sufficient feeder and/or antigen cells are not present at the onset of culture, desired cells may not expand. However, the cell composition can be altered to include an additional cell type acting as feeder cells and/or antigen presenting cell to allow expansion.

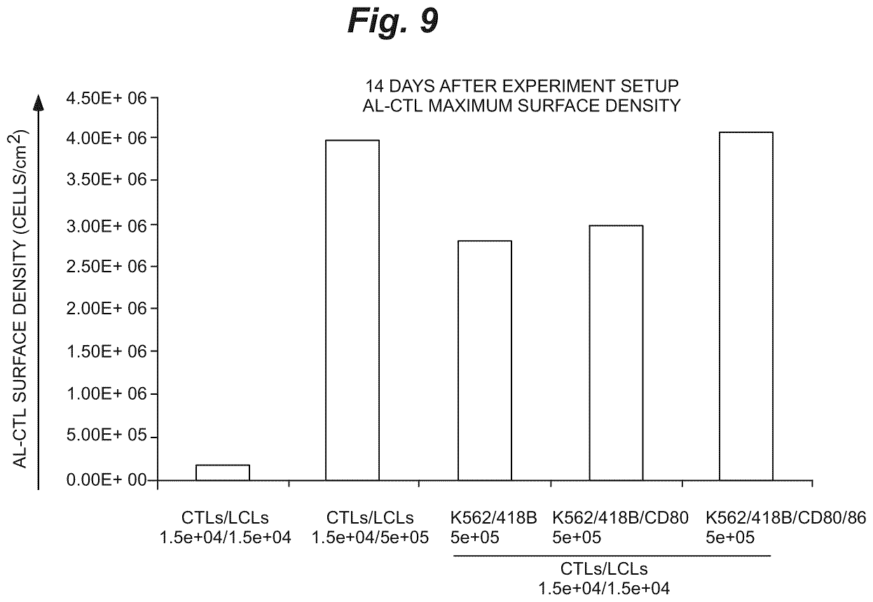

FIG. 9 shows an illustrative example in which we experimentally demonstrated that a very low cumulative surface density of desired cells and antigen presenting cells (in this case AL-CTLs and LCLs cells combining to create a cell composition with a surface density of 30,000 cells/cm²) was unable to initiate outgrowth of the AL-CTL population. However, this same cell composition could be made to grow by altering the composition to include another cell type acting as a feeder cell. In this case we evaluated a feeder layer of three various forms of irradiated K562 cells at a surface density of about 0.5×10⁶ cells/cm² and in all cases the population of AL-CTL expanded from the initial cell composition depicted in the first column of the histogram to move from a surface density of just 15,000 cells/cm² to a surface density of 4.0×10⁶ cells/cm² over 14 days. We also demonstrated, as opposed to the addition of a third cell type, increasing the population of LCLs achieved similar favorable results. The high surface density used for the LCL or K562 was arbitrarily chosen to demonstrate that a very low population of desired cells can be used to initiate growth when the cell composition includes an adequate number of feeder and/or antigen specific cells. When feeder cells are in short supply, expensive, or cumbersome to prepare, reducing their surface density to below 0.5×10⁶ cells/cm² is recommended. In general, and as we have demonstrated, when antigen presenting cells and/or feeder cells are in the cell composition, the additive surface density of the antigen presenting cells and/or feeder cells and the desired cells should preferably be at least about 0.125×10⁶ cells/cm² to create enough surface density in the cell composition to initiate the expansion of the desired cell population. Also, to attain the continued expansion beyond standard surface density limits, the use of growth surfaces comprised of gas permeable material was used in this example along with a medium volume to surface area ratio of 4 ml/cm².

### Example 8

Reduced desired cell surface densities, altered responder cell to stimulatory cell ratios, increased medium to growth surface area ratios, and periodic distribution of cells at a low surface density culture onto growth surfaces comprised of gas permeable material allow more desired cells to be produced in a shorter period of time and simplifies the production process when compared to other methods.

To further evaluate our ability to simplify and shorten the production of desired cells, we used G-Rex test fixtures for the initiation and expansion of EBV-CTLs. For purposes of this example, G-Rex500 refers to device as described in FIG. 6, the exception being the bottom is comprised of a 100 cm² growth surface area and a 500 ml medium volume capacity is available.

For the initial stage of EBV-CTL production, we seeded PBMCs in the G-Rex40 at a surface density of 1×10⁶/cm² (total=10⁷ PBMCs distributed over 10 cm² growth surface area of the G-Rex40) and stimulated them with EBV-LCL using a 40:1 ratio of PBMC:EBV-LCL. For CTL production, this 40:1 ratio is preferable in the first stimulation to maintain the antigen-specificity of the responder T cells. After the initial stage of culture, a second stage was initiated on day 9, wherein 1×10⁷ responder T cells were transferred from the G-Rex40 to a G-Rex500 test fixture. To initiate stage two of culture, 200 ml of CTL medium was placed in the G-Rex500,creating a medium volume to surface area ratio at the onset of stage two of 2 ml/cm² medium height at 2.0 cm above the growth surface area. The surface density of desired cells at the onset of stage two was 1×10⁵ CTL/cm² with antigen presenting cells at a surface density of 5×10⁵ LCL/cm², thereby creating a non-conventional 1:5 ratio of desired cells to antigen presenting cells. This stage two cell surface density and R:S ratio produced consistent EBV-CTL expansion in all donors screened. Four days later (day 13), IL-2 (50 U/ml-final concentration) was added directly to the culture, as was 200 ml of fresh medium, bringing medium volume to surface area ratio to 4 ml/cm².

On day 16, the cells were harvested and counted. The median surface density of CTLs obtained was 6.5×10⁶ per cm² (range 2.4×10⁶ to 3.5×10⁷).

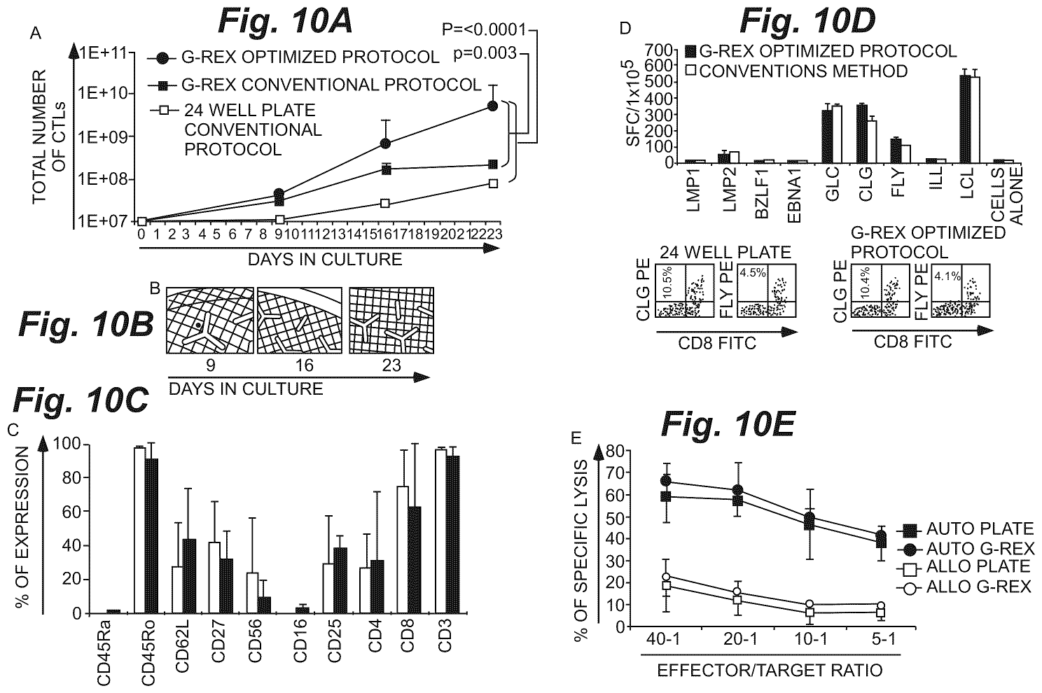

Compared to conventional protocols, the use of growth surfaces comprised of gas permeable material allows increased medium volume to surface area ratios (i.e. greater than 1 ml/cm²), lower cell surface densities (i.e. less than 0.5×10⁶/cm²), and altered ratios of responder to stimulator cells (less than 4:1) to create a decrease in production time. FIG. 10A shows the comparison of this G-Rex approach of Example 8 to the use of conventional methods of Example 1 and the G-Rex approach described in Example 5. As shown, the conventional method needed 23 days to deliver as many desired cells as could be delivered in either G-Rex method in about 10 days. After 23 days, the G-Rex approach of Example 8 was able to produce 23.7 more desired cells than the G-Rex method of Example 5 and 68.4 times more desired cells than the conventional method of Example 1. Furthermore, the desired cells continued to divide until day 27-30 without requiring additional antigen presenting cell stimulation provided the cultures were split when cell surface density was greater than 7×10⁶/cm².

Although the CTLs could not be viewed clearly in the G-Rex using light microscopy, clusters of CTLs could be visualized by eye or by inverted microscope and the appearance of the cells on days 9, 16, and 23 of culture is shown in FIG. 10B. Culture in the G-Rex did not change the phenotype of the expanded cells as shown in FIG. 10C, with greater than 90% of the cell composition being CD3+ cells (96.7±1.7 vs. 92.8±5.6; G-Rex vs. 24-well), which were predominantly CD8+ (62.2%±38.3 vs. 75%±21.7). Evaluation of the activation markers CD25 and CD27, and the memory markers CD45RO, CD45RA, and CD62L, demonstrated no substantive differences between EBV-CTLs expanded under each culture condition. The antigen specificity was also unaffected by the culture conditions, as measured by ELIspot and pentamer analysis. FIG. 10D shows a representative culture in which T cells stimulated with EBV peptide epitopes from LMP1, LMP2, BZLFI and EBNA1 and stained with HLA-A2-LMP2 peptide pentamers staining showed similar frequencies of peptide-specific T cells. Further, the expanded cells maintained their cytolytic activity and specificity and killed autologous EBV-LCL (62%±12 vs. 57%±8 at a 20:1 ET ratio; G-Rex vs. 24-well plate), with low killing of the HLA mismatched EBV-LCL (15%±5 vs. 12%±7 20:1 ratio) as evaluated by ⁵¹Cr release assays as shown in FIG. 10E.

Discussion of various novel methods for improved cell production for cell therapy: Examples 1-8 have been presented to demonstrate to skilled artisans how the use of various conditions including reduced surface density of the desired cell population at the onset of a production cycle, reduced surface density ratios between responder cells and stimulating cells, growth surfaces comprised of gas permeable materials, and/or increased medium volume to growth surface area ratios can be used to expedite and simplify the production of cells for research and clinical application of cell therapy. Although Examples 1-8 were related to the production of antigen specific T cells, these novel culture conditions can be applied to many important suspension cell types with clinical relevance (or required for pre-clinical proof of concept murine models) including regulatory T cells (Treg), natural killer cells (NK), tumor infiltrating lymphocytes (TIL), primary T lymphocytes, a wide variety of antigen specific cells, and many others (all of which can also be genetically modified to improve their function, in-vivo persistence or safety). Cells can be expanded with feeder cells and/or antigen presenting cells that can include PBMC, PHA blast, OKT3 T, B blast, LCLs and K562, (natural or genetically modified to express and antigen and/or epitope as well as co-stimulatory molecules such as 41BBL, OX40L, CD80, CD86, HLA, and many others) which may or may not be pulsed with peptide and/or a relevant antigen.

Unconventionally Low Initial Surface Density: One aspect of the present invention is the discovery that production time can be reduced relative to conventional methods by the use of lower desired cell surface density. In this manner, desired cells are able to have a greater numerical difference between their minimum and maximum cell surface densities than conventional methods allow. Preferably, when the rate of desired cell population growth has begun to diminish, but the quantity of desired cells is not yet sufficient to terminate production, the desired cells are re-distributed upon additional growth surfaces comprised of gas permeable material at low starting surface density once again.

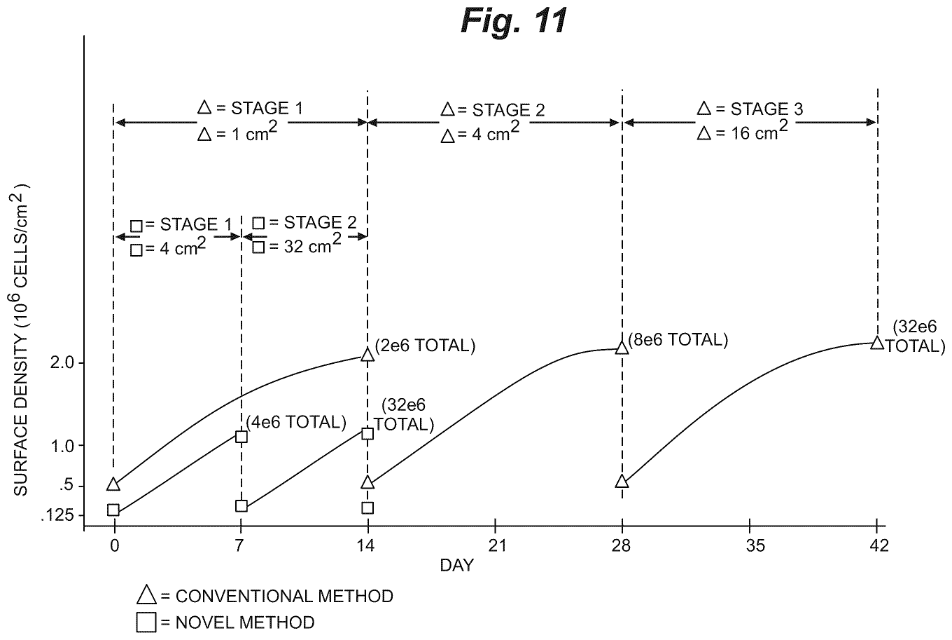

To explain how our novel cell production methods that rely upon lower surface density at the onset of any given culture stage can be applied, an example is now described. FIG. 11 shows a graphical representation of expansion of a desired cell population on a growth surface under the conventional scenario as compared to population expansion of the desired cell type using one aspect of the present invention. In this novel method, the surface density of desired cells at the onset of a production stage is less than conventional surface density. In order to make the advantages of this novel method the focus, this explanation does not describe the process of initially obtaining the desired cell population. The ‘Day” of culture starts at “0” to allow skilled artisans to more easily determine the relative time advantages of this novel method. In this example, each production cycle of the conventional method begins at a conventional surface density of 0.5×10⁶ desired cells/cm² while each production cycle of this example begins at a much lower and unconventional surface density of 0.125×10⁶ desired cells/cm². Thus, 4 times more surface area (i.e. 500,000/125,000) is required in this example to initiate the culture of than the conventional methods require. In this example, the desired cells of the conventional method reaches a maximum surface density of 2×10⁶ cells/cm² in 14 days. Thus, 1 cm² of growth area delivers 2×10⁶ cells/cm² which are then re-distributed onto 4 cm² of growth area so that production can be continued using the conventional starting density of 0.5×10⁶ cells/cm² (i.e. 4 cm² times 0.5×10⁶ cells=2×10⁶ cells). The cycle repeats for another 14 days at which point maximum cell surface density is again reached, with each of the 4 cm² of growth surface area delivering 2.0×10⁶ cells for a total of 8.0×10⁶ cells that are then distributed onto 16 cm² of growth area and the growth cycle repeats to deliver a total of 32×10⁶ cells over 42 days.

The novel method depicted in FIG. 11, instead of using the conventional method of depositing 500,000 desired cells onto 1 cm² at the onset of production, distributes the 500,000 cells equally onto 4 cm² of growth area to create at unconventionally low starting surface density of 125,000 desired cells/cm² on Day 0. In example the novel method, as with the conventional method, has its growth rate about to diminish on Day 7. Cells in the novel method are at a surface density of 1×10⁶ cells/cm². Thus, at the time point where growth rate is about to diminish, this stage of culture has produced 4×10⁶ cells that are then re-distributed onto 32 cm² of growth area so that production in Stage 2 can be continued using the starting surface density of 0.125×10⁶ cells/cm² (i.e. 32 cm² times 0.125×10⁶ cells=4×10⁶ cells). The cycle, or stage, of production repeats for another 7 days to Day 14, at which point maximum cell surface density is again reached, with each of the 32 cm² of growth surface area containing 1.0×10⁶ desired cells to yield a total of 32×10⁶ cells in just 14 days. Note how at the end of each production cycle, as with the conventional method, the novel method delivers a multiple of the finishing surface density divided by the starting surface density. However, by lowering starting cell surface density and completing each stage of production before cells have entered a growth production time is dramatically lowered. This example that describes how, by lowering the desired cell surface density (in this case to 0.125×10⁶ cells/cm²) relative to conventional cell surface density, the same quantity of desired cells are delivered in just 33% of the time as the conventional method (14 days vs. 42 days).

Although we quantified the advantages using a starting surface density of 0.125×10⁶ cells/cm², skilled artisans should be aware that this example of the present invention demonstrates that any reduction below conventional cell surface density will reduce production duration. Furthermore, skilled artisans will recognize that in this and other novel methods presented herein, the rate of cell growth and point at which diminished cell growth occurs described is for illustrative purposes only and the actual rates will vary in each application based on a wide variety of conditions such as medium composition, cell type, and the like. Additionally, for a given application, skilled artisans will recognize that the advantage of this aspect of the present invention is the production time reduction resulting from the reduction of cell surface density below that of conventional cell surface density in any particular application, wherein the particular conventional surface density used in this illustrative example may vary from application to application.

Thus, one aspect of the methods of the present invention when there is a desire to minimize the duration of production for a given quantity of desired cells that reside within a cell composition by use of reduced cell surface density is now described. Desired cells should be deposited upon a growth surface at an unconventionally low cell surface density such that:
a. the desired cells are in the presence of antigen presenting cells and/or feeder cells and with medium volume to surface area ratio of up to 1 ml/cm² if the growth surface is not comprised of gas permeable and up to 2 ml/cm² if the growth surface is comprised of gas permeable, and
b. the preferred surface density conditions at the onset of a production cycle being such that the target cell surface density is preferably less than 0.5×10⁶ cells/cm² and more preferably diminishing as described in FIG. 4, and
c. the surface density of the desired cells plus the surface density of the antigen presenting cells and/or feeder cells is preferably at least about 1.25×10⁵ cells/cm².

Based on the examples above, it is advisable for one to verify that the expansion of the desired cell population does not become limited if there is an attempt to further reduce the surface density of the antigen presenting cells and/or feeder cells below 1.25×10⁵ cells/cm². We selected 1.25×10⁵ cells/cm² based on the goal of demonstrating that outgrowth of a population of desired cells at unconventionally low density can be achieved when augmented by an adequate supply of antigen presenting cells and/or feeder cells.

Use of growth surfaces comprised of gas permeable material and higher medium volume to growth surface area ratios can simplify and shorten production. Another aspect of the present invention is the discovery that the use of growth surfaces comprised of gas permeable material and medium volume to growth surface area ratios that exceed conventional ratios, and repeated cycles of production that increase the amount of growth surface area used over time will reduce production duration.

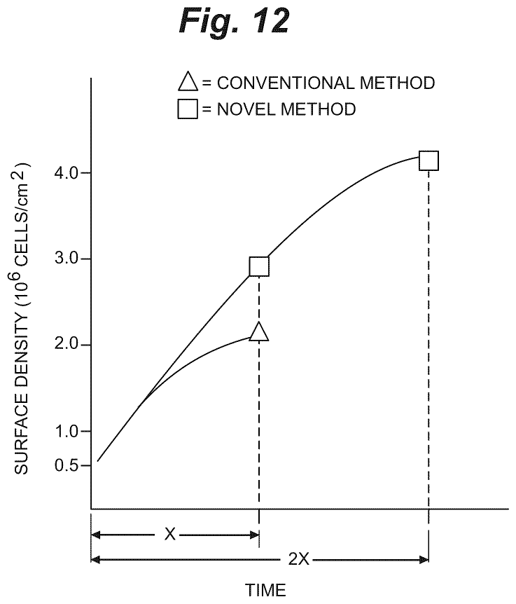

An illustrative example is now presented to show how these conditions can reduce the duration of production. FIG. 12 augments the discussion to show an example of the advantages that can be obtained by utilizing a growth surface comprised of gas permeable material and an unconventionally high medium volume to growth surface area ratio beyond 1 or 2 ml/cm². The discussion that follows is intended to demonstrate to skilled artisans how, by use of such a method, several options become available including reducing production time, reducing the amount of growth surface area used, and/or reducing labor and contamination risk. Skilled artisans will recognize that FIG. 12 and associated discussion is merely an example, and does not limit the scope of this invention.

The cell composition containing the desired cell population in this illustrative example is assumed to consume about 1 ml per “X” period of time. FIG. 12 shows two production processes, labeled “conventional method” and “novel method.” At the onset of growth, each process begins with desired cells at a surface density of 0.5×10⁶/cm². However, the growth surface of in the novel method is comprised of gas permeable material and medium volume to surface area ratio is 2 ml/cm² as opposed to the conventional method of 1 ml/cm². In time period “X”, the desired cell population of the conventional method has a reached a surface density plateau of 2×10⁶/cm² and is depleted of nutrients while the additional medium volume of the novel method has allowed growth to continue and desired cell surface density is 3×10⁶/cm². If the novel method continues, it reaches a surface density of 4×10⁶/cm². Thus, many beneficial options accrue. The novel method can be terminated prior to time “X” with more cells produced than the conventional method, can be terminated at time “X” with about 1.5 times more cells produced than the conventional method, or can continue until the medium is depleted of nutrients with 2 times many desired cells produced as the conventional method in twice the time but without any need to handle the device for feeding. In order for the conventional method to gather as many cells, the cells must be harvested and the process reinitiated, adding labor and possible contamination risk. Since cell therapy applications typically only are able to start with a fixed number of cells, the conventional method does not allow the option of simply increasing surface area at the onset of production.

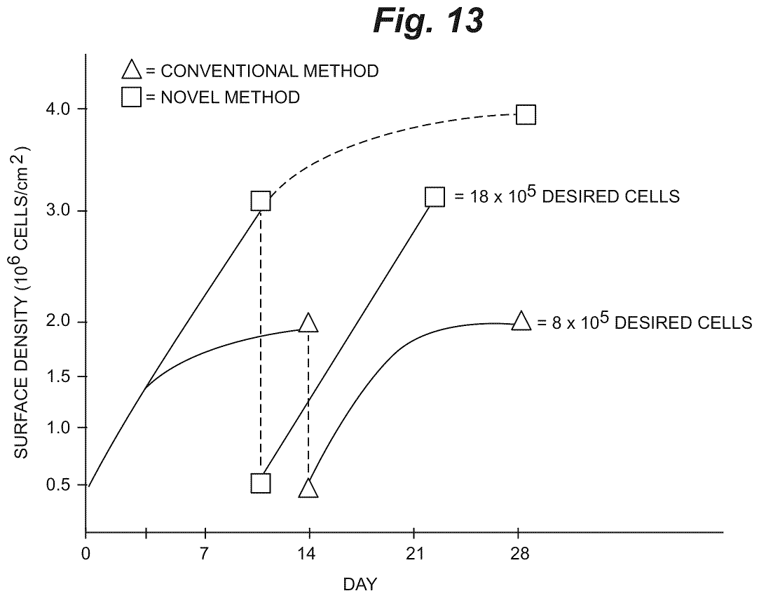

FIG. 13 continues the example of FIG. 12 to show how more than one production cycle can be of further benefit. FIG. 13 shows a graphical representation of expansion of a desired cell population on a growth surface under the conventional method as compared to population expansion of the desired cell type under one novel method of the present invention in which the surface density of the novel method exceeds surface density of the conventional method. In order to make this embodiment the focus, this explanation does not describe the process of obtaining the desired cell population. The ‘Day” of culture starts at “0” to allow skilled artisans to more easily determine the relative time advantages of this aspect of the invention. In this example, both cultures are initiated using conventional desired cell surface density of 0.5×10⁵ cells/cm² at “Day 0”. In this illustrative example, the growth surface of the conventional method is also comprised of gas permeable material. However, the medium volume to growth surface ratio in the conventional method is 1 ml/cm² as opposed to 4 ml/cm² in the novel method. As shown in FIG. 13, the desired cell population in the conventional method begins to diminish in growth rate when it is at a surface density of about 1.5×10⁶ cells/cm² in about 4 days and reaches a maximum surface density of 2×10⁶ cells/cm² in 14 days. At that point the desired cell population is distributed to 4cm² of growth area at a surface density of 0.5×10⁶/cm² in fresh medium at 1.0 ml/cm² and the production cycle begins again, reaching a surface density of 2×10⁶ cells/cm² in another 14 days and delivering 8×10⁶ desired cells in 28 days. By comparison, the desired cell population in the novel method begins to diminish in growth rate when it is at a surface density of about 3×10⁶ cells/cm2 in roughly about 10 to 11 days and could reach a maximum surface density of 4×10⁶ cells/cm2 in 28 days. However, to accelerate production, the cycle ends when the desired cell population is still in a high rate of growth. Thus, at about 10 to 11 days the 3×10⁶ cells are re-distributed to 6 cm² of growth surface area at a surface density of 0.5×10⁶/cm² in fresh medium at 4.0 ml/cm² and the production cycle begins again, with the desired cell population reaching a surface density of 3×10⁶ cells/cm² in roughly another 10 to 11 days and delivering 18×10⁶ desired cells around 21 days. Thus, in about 75% of the time, the novel method has produced over 2 times the number of desired cells as compared to the conventional method.

We have been able to obtain cell surface density in excess of 10×10⁶ cells/cm² upon growth surfaces comprised of gas permeable material, demonstrating that the use of the high surface density aspect of our invention is not limited to the density described in this example.

Thus, another example of the methods of the present invention when there is a desire to minimize the duration of production for a given quantity of desired cells that reside within a cell composition by use of reduced cell surface density is now described:
a. seeding the desired cells upon a growth surface area comprised of gas permeable material and in the presence of antigen presenting cells and/or feeder cells and with medium volume to surface area ratio of at least 2 ml/cm², and
b. establishing the preferred surface density conditions at the onset of a production cycle such that the target cell surface density is within the conventional density of about 0.5×10⁶ cells/cm², and
c. allowing the desired cell population to expand beyond the conventional surface density of about 2×10⁶ cells/cm², and
d. if more of the desired cells are wanted, redistributing the desired cells to additional growth surface comprised of gas permeable material and repeating steps a-d until enough desired cells are obtained.

When using these novel methods, further benefits can be attained by combining the attributes of initiating culture using unconventionally low surface area, using novel surface density ratios of desired cells and/or feeder cells, utilizing a growth surface area comprised of gas permeable material, utilizing unconventionally high ratios of medium volume to growth surface area, and conducting production in cycles. The conditions can be varied at any cycle of production to achieve the desired outcomes, such as striking a balance between reduced production time, surface area utilization, feeding frequency, and the like.

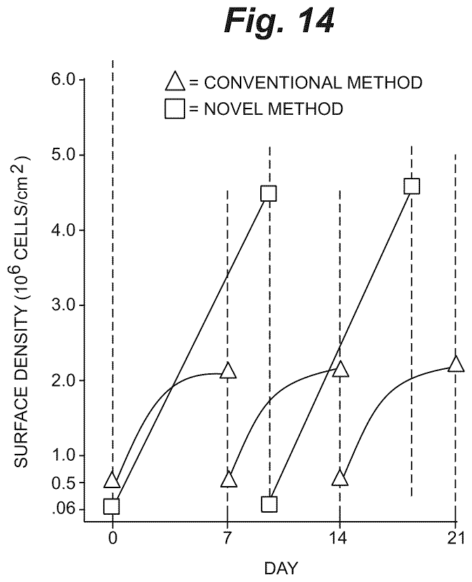

FIG. 14 shows another novel method in which still further advantages relative to conventional methods are obtained. As with other illustrative embodiments described herein, skilled artisans will recognize that the description herein does not limit the scope of this invention, but instead acts to describe how to attain advantages of improved production efficiency.

In this example, desired cells are doubling weekly in conventional conditions. The ‘Day” of culture starts at “0” to allow skilled artisans to more easily determine the relative time advantages of this embodiment. Also, issues previously described related to feeder and/or antigen presenting cell surface density ratios are not repeated to simplify this example. For illustrative purposes, assume a starting population of 500,000 desired cells with a doubling time of 7 days in conventional conditions is present on “day 0” production. The conventional method begins with a surface density of 0.5×10⁶ cells/cm² and a medium volume to surface area ratio of 1 ml/cm². As shown, when the population of the desired cells reaches a surface density of 2×10⁶ cells/cm² the cells are distributed onto additional surface area at a surface density of 0.5×10⁶ cells/cm² and the production cycle begins anew. The novel method of this example begins with a surface density of 0.06×10⁶ cells/cm², a growth surface area comprised of gas permeable material, and a medium volume to surface area ratio of 6 ml/cm². As shown, when the population is nearing the start of a growth plateau, cells are redistributed to more growth surface area. In this case, the population is determined to be reaching plateau from noting that plateau is initiated in the conventional method when cell surface density approaches 1.5 times the medium volume to surface area ratio (i.e. about 1.5×10⁶ cells/ml). Thus, at a surface density of about 4.5×10⁶ cells/cm² at about 9 days, cells are distributed onto 36 cm² of growth surface area and the production cycle begins anew.

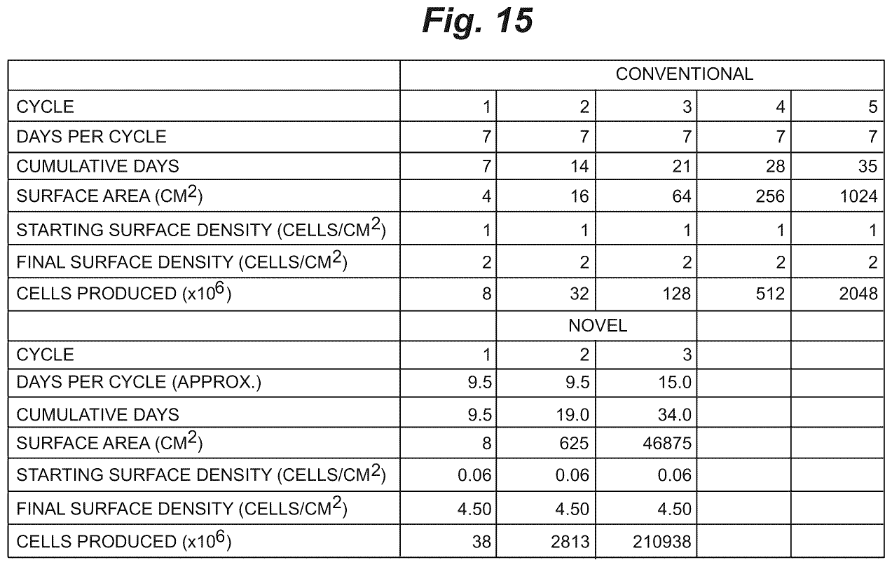

FIG. 15 tabulates a comparison of each production method depicted in FIG. 14, and extends to stages to demonstrate the power of the novel method, and why it is wise to adjust the production protocol at various stages to fully capture the efficiency. Note that the novel method overpowers the conventional method after completing just the second stage of the production cycle, delivering nearly 1.37 times more cells in only about half the time with just 61% of the surface area requirement. However, note how the third stage of the production cycle creates a massive increase in cells and a corresponding increase in surface area. Thus, one should model the production cycles to anticipate how to adjust the initial cell surface density and/or final cell surface density throughout each cycle of the process to attain an optimal level of efficiency for any given process.

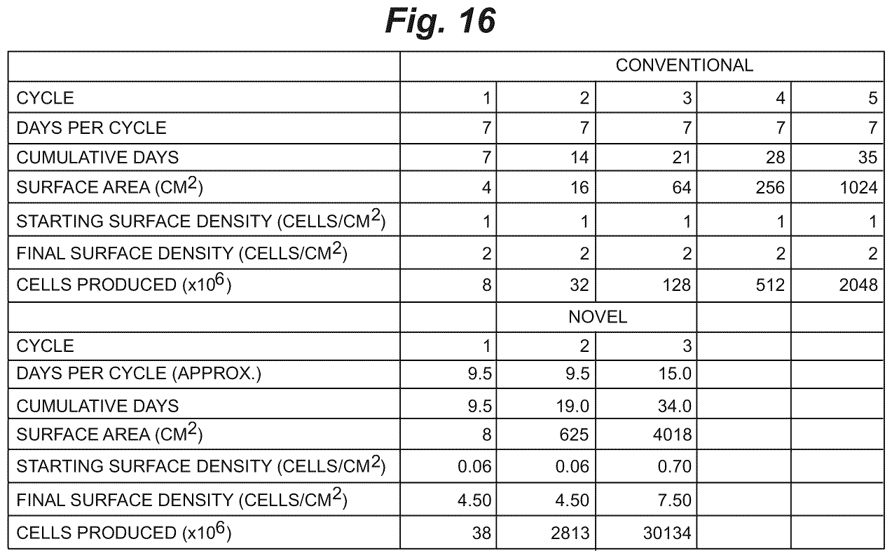

As an example, FIG. 16 shows an example of how one could alter variables in the novel method to gain efficiency as production progresses. For example, an increase in the starting surface density of cycle 3 from 0.06 to 0.70 cell/cm² and a change to the final surface density from 4.5 to 7.5 cells/cm² can be undertaken. Increasing the final surface density is a matter of increasing the medium volume to surface area ratio beyond the initial 6 ml/cm² to a greater number. The greater the medium volume to surface area, the longer the cycle remains in rapid growth phase (i.e. the population expansion prior to plateau). In this case we have allowed 5 extra days to complete the rapid growth phase and raised the medium volume to surface area ratio to about 8 ml/cm². So doing, in this example, allows over 3 trillion cells to be produced in 34 days with a reasonable surface area. For example, we have fabricated and tested devices with about 625 cm² of growth surface comprised of gas permeable material. This is clearly a superior approach to producing cells than the conventional method.

Thus, another preferred embodiment of the methods of the present invention when there is a desire to minimize the duration of production for a given quantity of desired cells that reside within a cell composition by use of reduced cell surface density is now described:
a. seeding the desired cells upon a growth surface area comprised of gas permeable material and in the presence of antigen presenting cells and/or feeder cells and with medium volume to surface area ratio of at least 2 ml/cm², and
b. establishing the preferred surface density conditions at the onset of a production cycle such that the target cell surface density is less than the conventional density, preferably at between about 0.5×10⁶ desired cells/cm² and about 3900 desired cells/cm² and total number of desired cells and antigen presenting cells and/or feeder cells being at least about 1.25×10⁵ cells/cm², and
c. allowing the desired cell population to expand beyond the conventional surface density of about 2×10⁶ cells/cm², and
d. if more of the desired cells are wanted, redistributing the desired cells to additional growth surface comprised of gas permeable material and repeating steps a-d until enough desired cells are obtained.

Disclosures of the present invention advance the field of Adoptive Cell Therapy by creating a new class of therapeutic cells called T-Vehicles. T-Vehicles are comprised of a population of T cells that do not carry inherent risk of GVHD, further altered to include one or more therapeutic attributes capable of acting with a therapeutic purpose in order to provide recipients with a therapeutic benefit. Since T-Vehicles do not have a native capacity to initiate GVHD disease, they become an ideal biological transportation vehicle to arm with any number of weapons capable of fighting a wide variety of medical conditions and diseases. The present invention discloses methods for producing and using T-Vehicles that are armed with therapeutic attributes for the purpose of providing recipients the health benefits of Adoptive Cell Therapy without inherent risk of GVHD that is present in state-of-the-art methods. Of importance, T-Vehicles function contrary to state-of-the-art methods for Adoptive Cell Therapy, as the therapeutic purpose of T-Vehicles is wholly unrelated to the native T cell receptor's antigen specificity. Skilled artisans are encouraged to recognize throughout the disclosures and illustrative embodiments presented, the therapeutic attribute of T-Vehicles does not include the native antigen receptors of the T-Vehicles.

T-Vehicles are produced by stimulating donor PBMCs or donor cord blood with antigen in order to activate growth of donor T cells that have native antigen specificity to the antigen, thereby producing an antigen-specific T cell population that comprises antigen receptors with antigen specificity to the antigen. By selecting antigens that are not present on normal cells, a population of T cells with antigen receptors that are not able to recognize normal cells can be created. By ignoring the therapeutic benefit that may derive from the antigen specificity of the native T cells, and altering the native T cells with therapeutic attribute(s) that do not include the native antigen receptors recognition capacity, a population of T-Vehicles can be created that have a purpose independent of their antigen specific recognition and are not inherently prone to, or even capable of, initiating GVHD.

Although T-Vehicles may encompass more than one population of native antigen-specific T cells, since T-Vehicles do not rely on their native antigen specificity for its therapeutic purpose, T-Vehicles can be infused into a recipient independent of whether or not the serotype of the recipient exhibits a positive match to any of the native antigen receptor(s) of T-Vehicles. Also, key attributes of T-Vehicles include their ability to be used in a HLA mismatched setting or, since the native T cell population(s) from which T-Vehicles are derived do not carry inherent risk of GVHD. This allows allogeneic banks of T-Vehicles to be established that can service a wide segment of society without the limitations of HLA matching that is required in state-of-the-art methods. When T-Vehicles have native antigen specificity that is a HLA mismatch to the recipient, native T cells receptors of the T-Vehicles are incapable of recognizing cells in the recipient and initiating GVHD. Nevertheless, T-Vehicles commence with their therapeutic activity in a completely HLA mismatched setting because they have been altered with therapeutics attributes that do not rely on the native antigen receptors to accomplish its therapeutic purpose. T-Vehicles are not limited to use in HLA mismatched setting however. By creating T-Vehicles comprised of T cells that have native antigen receptors with highly restricted antigen-specificity against antigens not expressed on normal cells, the initiation of GVHD disease can be avoided despite a partial HLA match between the recipient and the native antigen specificity of the T-Vehicle. To allow T-Vehicles to be used in HLA matched or HLA mismatched settings, it is preferable that the native antigen specificity of the T-Vehicles only allows them to recognize antigens that are not present on normal cells, more preferably normal human cells, even more preferably are only able to recognize a single epitope of antigens that are not present on normal mammalian cells.

In the event T-Vehicles are a HLA mismatch to the recipient, the recipient is expected to mount a vigorous immune response that will eventually eliminate the T-Vehicles. Therefore, the therapeutic purpose of the T-Vehicles can be continued by the delivery of one or more additional doses of T-Vehicles. This process can continue as needed to obtain the desired therapeutic purpose. In the preferred method, each dose of T-Vehicles differs in HLA so that the patient's immune system needs to re-prime itself each time it prepares to attack a new dose of T-Vehicles, thereby keeping the interval between each dose of T-Vehicles roughly equal.

Methods of producing T cells that have native antigen receptors with highly restricted antigen-specificity: Historically, producing populations of T cells at the scale needed for wide spread use in Adoptive Cell Therapy has been virtually impossible. State-of-the-art production methods for expanding T cells populations into suitably sized therapeutic doses are so impractical and unmanageable that they limit cell therapy to just very small population that must be treated at a small number of highly specialized institutes. A fundamental attribute of T-Vehicles is that their native T cell characteristics do not inherently expose the recipient to GVHD. Since it preferable that the native antigen specificity of the T-Vehicles only allows them to recognize antigens that are not present on normal cells, more preferably normal human cells, even more preferably are only able to recognize a single epitope of antigens that are not present on normal mammalian cells, efficient production of these cells becomes a cornerstone for wide spread use of methods involving T-Vehicles. Such T cells are only present at very low, and sometimes undetectable, frequencies in donor PBMCs or cord blood. Thus, the problems inherent to state-of-the-art T cell production methods are compounded when trying to generate populations of T cells that are most suitable for use in T-Vehicles.

We have discovered methods and apparatus, as described in U.S. patent application Ser. No. 13/475,700, filed May 18, 2012, entitled “IMPROVED METHODS OF CELL CULTURE FOR ADOPTIVE CELL THERAPY (hereinafter referred to as Vera '700), and which is incorporated by reference herein, that contradict state-of-the-art methods in order to efficiently produce T cells with native characteristics that do not inherently expose the recipient to GVHD. In so doing, the long standing need for practical production of T cells found at low frequencies in donor PBMCs or cord blood is met. Moreover, when combined with the novel concept of T cells that possess therapeutic attributes that are not inherent to the native antigen specificity of the T cell, the production of T-Vehicles that act as biological carriers not only becomes possible, it becomes practical.

In one illustrative method, at the onset of culture more than one selected antigen is presented to PBMCs or cord blood (i.e. the original pool of antigen specific T cells) with the intention of stimulating outgrowth of more than one unique antigen-specific T cell population (each population expressing an antigen receptor to one of the antigens presented). The intent is to subsequently select the most prolific and/or desirable native T cell population for production and terminate the others. As the culture proceeds after onset, the various T cell populations responding to the various antigens are likely to exhibit differing levels of population expansion, depending on the magnitude of their original population. Furthermore, some or all may continue to be undetectable. After some time, the culture is assessed for acceptable outgrowth of T cell populations reacting to any of the selected antigens. Such an assessment could be for just one population specific to one antigen, or to additional populations specific to additional antigens. If one antigen-specific T cell population is demonstrating acceptable expansion, re-stimulating that particular T cell population by only adding the antigen it recognizes into the device will cause the remaining T cells to eventually die, while the particular desired T cell population continues to proliferate. However, if more than one T cell population is demonstrating acceptable expansion, there are two options 1) the culture can be re-stimulated with only the antigens those particular T cell populations are reacting to (thereby terminating expansion of less prolific T cell populations) or 2) the culture can split into more than one culture device, each device receiving a single antigen differing from all other devices antigen thereby causing only one T cell population to proliferate in each device with all but the most prolific cultures eventually being terminated. Preferably, all culture devices are gas permeable and of the types described in co-pending U.S. Publication Nos. 2005/0106717 A1 to Wilson et al. (hereinafter referred to as Wilson '717) and 2008/0227176 A1 to Wilson (hereinafter referred to as Wilson '176), which are both incorporated by reference herein, and rely on the methods of Vera '700.

By way of additional example, a population of PBMCs residing in a culture device could be presented with antigen A, antigen B, and antigen C. After period of time, the culture could be assessed for the presence and/or proliferation of populations reactive to antigens A, B, or C. If an antigen specific population reactive to antigen A is the only population not exhibiting acceptable frequencies and/or population expansion, it can be terminated by re-stimulation with only antigen B and antigen C. Alternatively, if antigen specific population reactive to antigen B and antigen C were proliferating about equally, but it was uncertain which would continue to proliferate the at best rate, the culture could be split into two devices with the expectation that one device would eventually continue production while the other would be terminated. The first device would receive antigen B and the second device would receive antigen C. T cells exhibiting antigen specificity to antigen B would proliferate in the first device but T cells exhibiting antigen specificity to antigen C eventually would die off. Vice versa in the second device. At some point in time after the onset of culture in the first and second devices, examination of the frequency and/or population size could be undertaken with the intent of terminating the culture with the least efficient expansion of the desired T cell population. Skilled artisans are encouraged to recognize that a primary advantage of initiating culture with multiple antigens at onset, as opposed to just one antigen, is that it increases the prospects of finding a T cell population of suitable antigen specificity and growth rate. Furthermore, using multiple antigens in one device instead of multiple devices with one antigen makes more efficient use of PBMCs or cord blood, medium, cytokines, laboratory space, labor, and bio-hazardous disposal space.

Selecting the preferred native antigen specificity of T-Vehicles is now described: Although it is preferable that the native antigen specificity of the T-Vehicles only allows them to recognize antigens that are not present on normal cells, more preferably normal human cells, even more preferably are only able to recognize a single epitope of antigens that are not present on normal mammalian cells, this is non-limiting and there are many suitable attributes of the native antigen receptors skilled artisans are encouraged to consider. Many options and characteristics are suitable. As examples, the native antigen specificity of T-Vehicles can be composed of more than one population of T cells with native antigen specificity. The native antigen specificity of the T-Vehicles can be against a whole antigen or a single epitope of self or a non-self antigens; reptiles, amphibians, fish, or birds; invertebrates such as sponges, coelenterates, worms, arthropods, mollusks, or echinoderms; bacteria, fungus, parasites, and sponges; viruses including but not limited to adenovirus, Epstein-Barr virus (EBV), Cytomegalovirus (CMV), Adenovirus (Adv), Respiratory Syncytial virus (RSV), human herpesvirus 6 (HHV6), human herpesvirus 7 (HHV7), BK virus, JC virus, Influenza, H1N1, parainfluenza, herpes simplex virus (HSV), Varicella Zoster Virus (VZV), Parvovirus B19, Coronavirus, Metanpneumovirus, Bocavirus, or KI virus/WU virus; or Survivin, gp 100, tyrosinase, SSX2, SSX4, CEA, NY-ESO-1, PRAME, MAGE-A1, MAGE-A3, MAGE-A4, Claudin-6, Cyclin-B1, Her2/neu-ErbB2, Histone H1.2, Histone H4, Mammaglobin-A, Melan-A/MART-1, Myc, p53, ras, PSA, PSMA, PSCA, Sox2, Stromelysin-3, Trp2, WT1, Proteinase 3, Muc 1, Alphafetoprotein, CA-125, bcr-abl, hTERT, or Prostatic Acid Phosphatase-3.

To facilitate the outgrowth of appropriate native T cell populations of donor cells, skilled artisans are encouraged to review U.S. Publication No. 2011/0182870 A1 (hereinafter referred to as Leen '870), and which is incorporated by reference herein, and also consider stimulation using antigen presenting cells (APCs) such as Dendritic cells, Monocytes, Macrophages, B cells, T cells, PBMCs or artificial antigen presenting cells such as engineered k562, any of which are able to present the desired antigens to produce the desired antigen specificity of the native donor T cell population and thus the native antigen specificity of the T-Vehicles; use of antigen for the induction of the desired immune response in the donor cells by use of cell lysate containing the desired antigen, purified protein containing the desired antigen, recombinant protein containing the desired antigen, plasmid DNA encoding for the desired antigen, plasmid RNA encoding the desired antigen describe, and/or peptide library containing the desired antigen, and/or single synthetic peptide(s) containing the desired antigen.

Production of the T cell population is preferably undertaken using the methods of Vera '700, and/or those presented herein, and most preferable they are undertaken utilizing gas permeable culture devices of the types described in Wilson '717 and/or Wilson '176. Skilled artisans are encouraged to recognize that various methods in the described body of work may be more or less appropriate depending on the specific objectives of each application. For example, various surface densities, medium heights, medium volume to growth surface areas and the like can be utilized, as well as stimulation with cytokines such as IL2, IL15, IL21, IL12, IL7, IL27, IL6, IL18 and/or IL4 and various frequencies and concentration, and use of repetitive in vitro stimulation using any source of antigen in combination with any of the methods of presenting the antigen is possible and can be undertaken with or without cell sorting by methods including by not limited to gamma capture, magnetic isolation, single cell cloning, and/or flow cytometry.

### Example 9

T-Vehicles with native T cell receptors recognizing the CMV epitope NLV are unable to recognize non-autologous cell targets.

Antigen specific T cells with native antigen specificity to NLV-CMV were expanded from a frequency of 0.03% in PBMCs to 87% in 12 days using the methods previously described. These cells were then placed in culture with cells from three HLA mismatched donors presenting the target CMV antigen's NLV peptide.

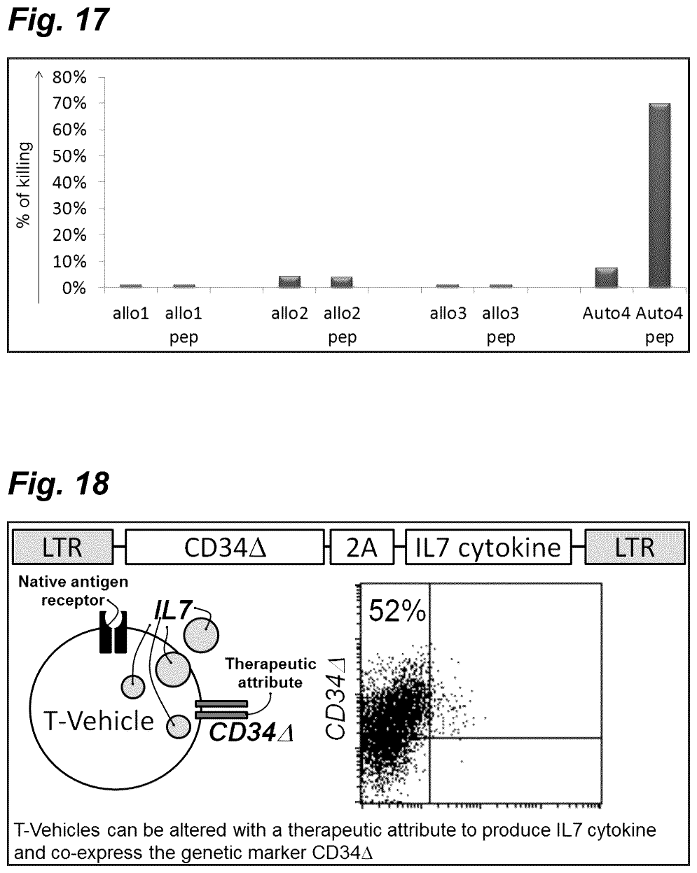

FIG. 17 shows how the T-Vehicles were unable to recognize cells from mismatched allogeneic donors whether or not they expressed the NLV peptide (“allo1” and “allo1 pep”, “allo2” and “allo2 pep”, “allo3” and “allo3 pep”) despite their full functionality as demonstrated by the capacity to recognize and kill autologous cells presenting the NLV peptide (“Auto4 pep”) and avoid killing autologous cells not presenting the NLV peptide (“Auto4”).

Selecting and creating the desired therapeutic attribute(s): There are a wide variety of options for altering the antigen specific T cell population to include at least one therapeutic attribute. Examples follow that are non-limiting, but intended to provide skilled artisans with recognition of how the choice of therapeutic attribute depends on the therapeutic purpose and why the therapeutic attribute and its therapeutic purpose are independent of the antigen specificity of the T-Vehicles native antigen receptors.

### Example 10

### T-vehicles Loaded With Recombinant Proteins Administered as an Adjuvant With Immunotherapies

Immunotherapies are a class of therapies which are designed to elicit or amplify an immune response in a patient. Examples including administration of vaccines designed to activate an immune response directed against tumor antigens expressed on cancer cells or delivery of ex vivo expanded T cells or NK cells. Recombinant proteins such as cytokines like IL2, IL7, GM-CSF, have been administered systemically in order to promote the growth, expansion, persistence and/or function of these cells in vivo but the systemic administration of some cytokines (e.g. IL2) has been associated with in vivo toxicity including severe mucositis, nausea, diarrhea, edema, respiratory distress, liver and renal dysfunctions, and the expansion of regulatory T cells that impair the function of the induced/infused T cells. Administration of T-vehicles loaded with recombinant proteins including cytokines can overcome such toxicities by migrating to the site of inflammation, and delivering these recombinant proteins directly at the site of inflammation (induced by the immunotherapy).

Skilled artisans are encouraged to recognize that T-vehicles can be used to target the delivery of such cytokines instead of the traditional unspecific systemic administration. For example, experiments were undertaken to create T-Vehicles able to produce the cytokine IL7 and to express a truncated form of CD34Δ which can be used to detect the percentage of transduce cells as wells as selecting the transgenic population. In this case, as shown in FIG. 18, donor T cells with 98% native antigen specificity for the NLV epitope of CMV virus were successful altered to create T-Vehicles with the therapeutic attribute of CD34Δ-IL7 cytokine expression as determine by flow analysis. Further testing demonstrated that only T-Vehicles modified with the retroviral vector (CD34Δ-IL7 cytokine) were capable of producing IL7, as detected by ELISA.

To evaluate the therapeutic T-Vehicles modified with the retroviral vector (CD34Δ/IL7cytokine), in terms of in-vivo effect and in-vivo distribution of the IL7 cytokine, mice were divided into two groups (5 animals per group). In Group 1, tumor bearing mice were treated with 2000 ng of IL7 cytokine administered systemically by IV. In Group 2, mice were treated with a single IV injection of 10E+06 T-Vehicles. Random subjects from each group were then sacrificed at week 1 and week 2 to evaluate by ELISA the IL7 cytokine concentration at different locations including the heart, liver, kidney, spleen, peritoneum, tumor and blood.

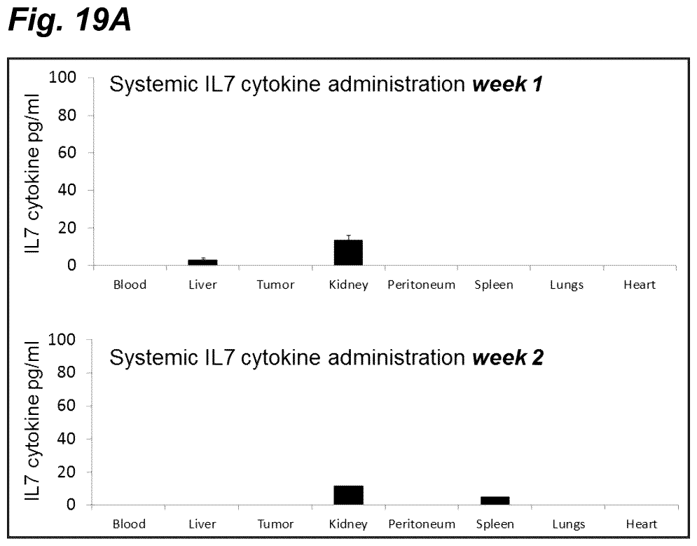

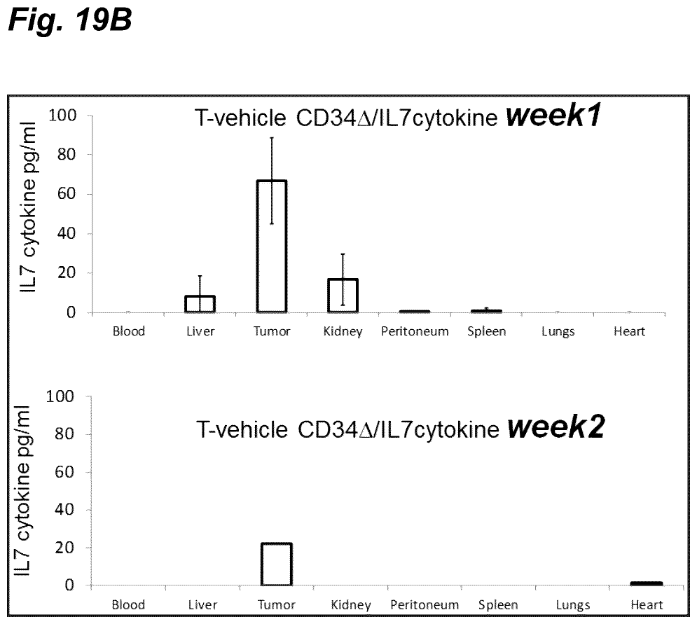

FIG. 19A shows the IL7 cytokine accumulation in the various locations for Group 1. The IL7 cytokine ELISA analysis demonstrate that higher cytokine levels were detected on the kidney and they were below detection at the tumor site.

FIG. 19B shows the IL7 cytokine accumulation in the various locations for Group 2. The IL7 cytokine ELISA analysis demonstrate greater cytokine concentration at the tumor site when compare with other organs and cytokine production was sustained at the tumor for at least 2 weeks after the administration of the T-vehicles. Therefore, T-Vehicles were able to migrate to the tumor site and preferentially deliver the cytokine IL7 for a sustained period of time. This clearly demonstrates the ability of the T-vehicles, with a therapeutic attribute capable of delivering cytokine, provides superior therapeutic benefits when compared state-of-the-art methods of cytokine delivery that are administered systemically. As expected, T-Vehicles have a limited in-vivo presence, as indicated by the reduction in cytokine concentration from week 1 to week 2. This can be viewed as an additional benefit of T-Vehicles, as they do not remain in the recipient. Preferably, additional doses of T-Vehicles would be administered as needed until therapeutic outcome is met, and without additional doses, the T-Vehicles would be purged from the recipient

### Example 11

Donor T cells can be modified to create T-Vehicles with the therapeutic attribute being a chimeric antigen receptor (CAR) that targets a particular antigen.

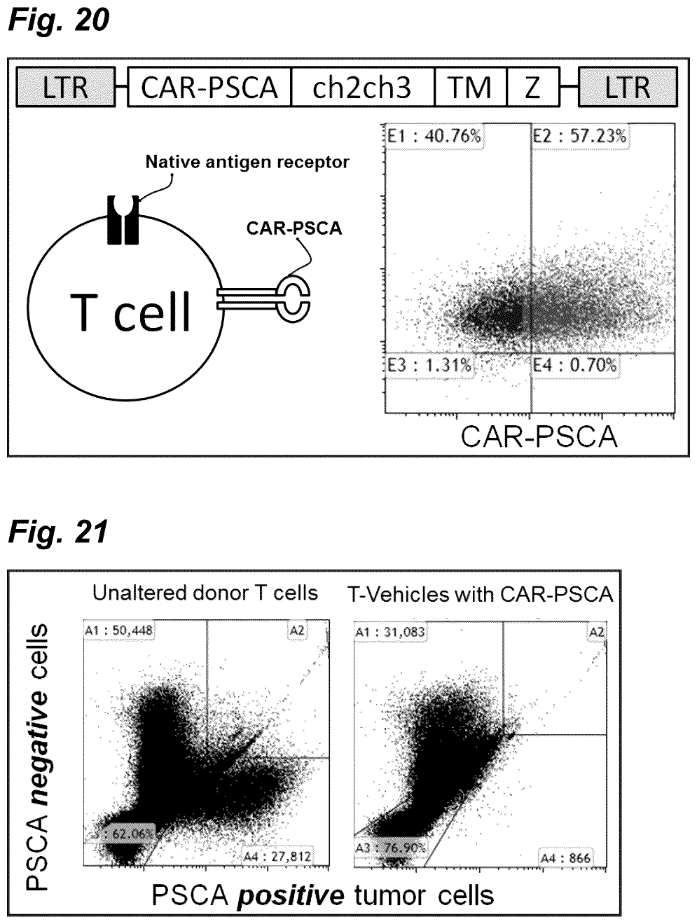

Donor T cells with 98% of T cells with native antigen specificity for the epitope NLV of the virus CMV (as evaluated by pentamer analysis) where transduced to create T-Vehicle with the therapeutic attribute of expressing CARs capable of recognizing prostate stem cell antigen (PSCA). The therapeutic purpose of the T-Vehicle is the destruction of prostate tumor cells. As depicted in FIG. 20, quadrant E2, 57.23% of the donor T cells with were successful altered to create T-Vehicles with the therapeutic attribute of CAR-PSCA as determined by flow analysis. To test the killing effectiveness of T-Vehicles including the therapeutic attribute of CAR-PSCA, unaltered donor T cells and T-Vehicles created by modifying the donor T cells with CAR-PSCA were cultured at a 1:1 ratio with target cells that were positive for the antigen PSCA (GFP+) or PSCA negative (mOrange+) and after 72 hours of culture, the number of residual PSCA positive tumor cells was quantified by flow analysis. FIG. 21 shows experimental outcomes at 72 hours, where quadrant Al represents the number of PSCA negative cells, quadrant A3 represents the number of T-vehicles, and quadrant A4 represents the number of PSCA positive tumor cells. As expected, after 72 hours the unaltered donor T cells did not alter the original culture composition. To the contrary however, T-vehicles expressing CAR-PSCA were able to nearly eradicate the entire population of PSCA positive tumor cells, while simultaneously demonstrating exquisite selection for the PSCA antigen by leaving the PSCA negative cells unharmed. This clearly demonstrates the T-Vehicles capacity to create a therapeutic benefit unrelated to its native antigen specificity.

### Example 12

Donor T cells can be altered to create T-Vehicles with the therapeutic attribute being a receptor that is capable of depleting unwanted cytokines in the recipient.

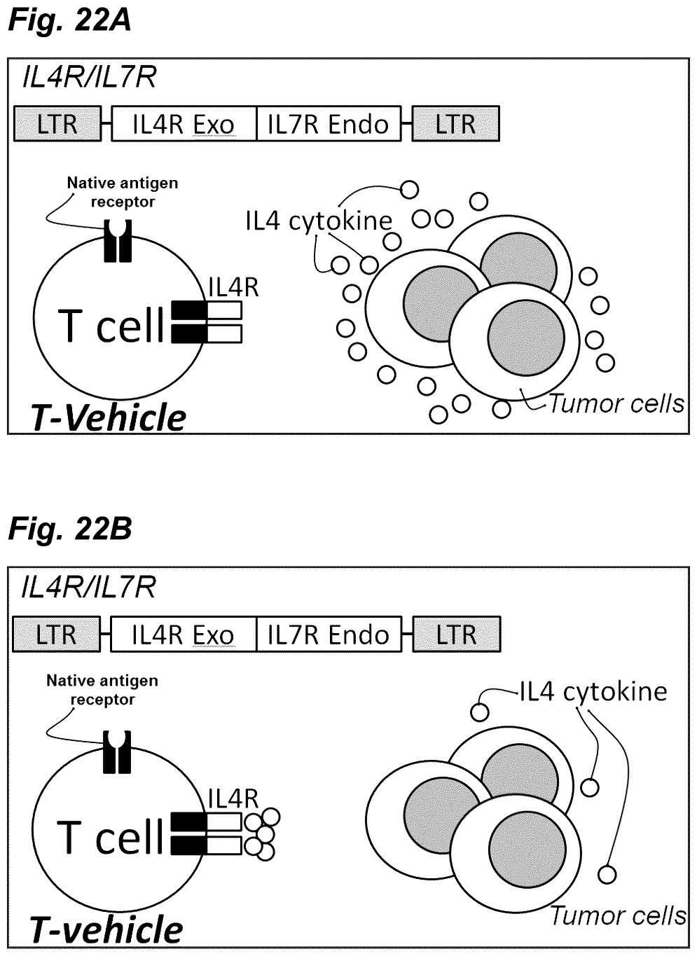

Tumor cells protect from the immune system by the production of immune-suppressive cytokines which repress the anti-tumor effect of the endogenous T cells. Donor T cells can be altered to create T-vehicles with the therapeutic attribute of expressing whatever particular cytokine receptors are needed to provide the therapeutic purpose of vacuuming the unwanted particular cytokines from the tumor, thereby having the therapeutic benefit of making the tumor environment more permissive to immunotherapy strategies. FIG. 22A and FIG. 22B show a representation of such a process. In the depiction of FIG. 22A, T-Vehicles including the therapeutic attribute of receptors capable of binding IL4 are in proximity of tumor cells expressing IL4 cytokine. In the depiction of FIG. 22B, the T-Vehicles have bound IL4 cytokines and the quantity of IL4 cytokines protecting the tumor cells is greatly reduced. Note how the therapeutic attribute, the therapeutic purpose, and the therapeutic benefit of the T-Vehicle does not include, and is independent of, the native antigen receptor of the T-Vehicle.

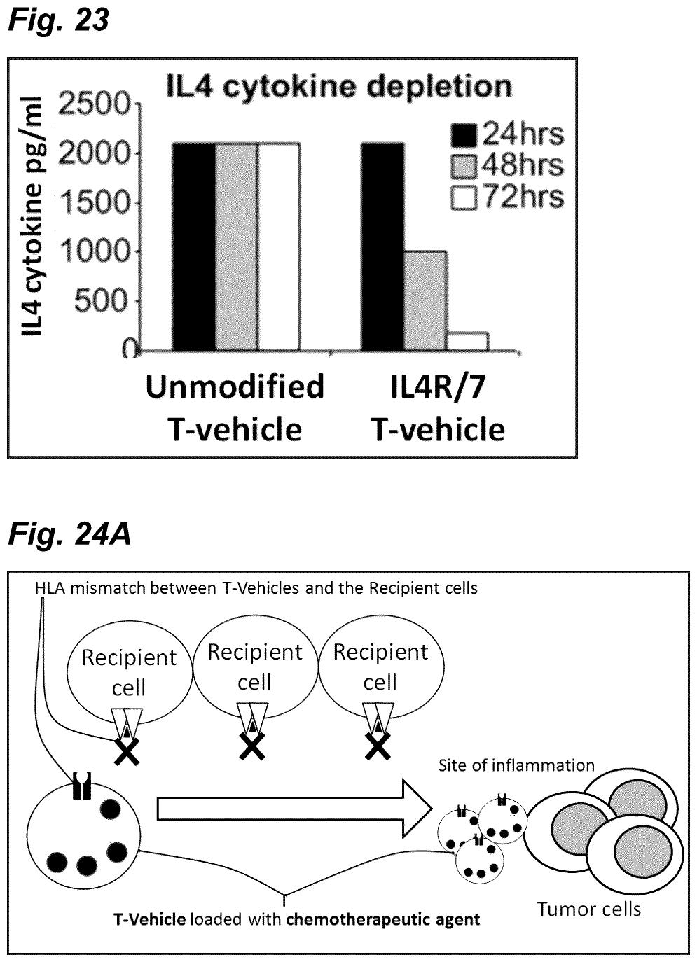

Experiments were conducted to evaluate the capacity of T-Vehicles, having a therapeutic attribute of expressing extra-cellular recombinant cytokine receptors IL4R/7, to deplete IL4 cytokine. T-Vehicles were prepared by altering donor T cells with native specificity for the NLV epitope of the CMV virus. 5E+05 T-Vehicles were culture in a 24 well plate in a volume of 2 mls of media in the presence of 2000 pg/ml of IL4 and compared the donor T cells. The concentration of the cytokine IL4 was then evaluated by ELISA at 24, 48 and 72 hs. Results are shown in FIG. 23. Clearly the T-Vehicles were able to meet their therapeutic purpose, as the reduction of the immune-suppressive tumor growth factor IL4 cytokine over a 72 hour period was striking. To the contrary, donor T cells (i.e. the histograms labeled “Unmodified T-vehicle”) showed no capacity to reduce the presence of IL4.

Skilled artisans are encouraged to recognize that there are many therapeutic attributes T-Vehicles can be equipped with in order to become capable of meeting a therapeutic purpose intended to provide a recipient with a therapeutic benefit. The disclosed possibilities are now augmented by several additional examples.

T-Vehicles altered with the therapeutic attribute of chemotherapeutic agents for the targeted treatment of cancer: A variety of different chemotherapeutic agents or anti-neoplastic drugs are used to treat different types of cancers including breast, prostate, pancreatic, liver, lung, brain, leukemia, lymphoma, melanoma, and myeloma. Most chemotherapy is delivered intravenously, although a number of agents can be administered orally, and subsequently circulates throughout the body. Chemotherapy agents act by killing cells that divide rapidly, one of the main properties of most cancer cells. This means that chemotherapy also harms cells that divide rapidly under normal circumstances (e.g. cells in the bone marrow, digestive tract, and hair follicles). This results in the most common side-effects of chemotherapy are myelosuppression (decreased production of blood cells, hence also immunosuppression), mucositis (inflammation of the lining of the digestive tract), and alopecia (hair loss). Chemotherapy-induced nausea and vomiting are also frequent side effects of treatment. Administration of T-Vehicles loaded with these drugs has the potential to offset these toxicities. This can occur by loading T-Vehicles with a chemotherapeutic agent, infusing them into a recipient, whereby they will migrate to sites of inflammation (cancer) down a chemotactic gradient. In this manner, the chemotherapeutic agent is placed in proximity of the tumor cells as opposed to being administered in a systemic manner to the recipient. In the case of an HLA mismatch, the recipient immune system will mount an attack on the T-Vehicles, causing them to be destroyed, but not without releasing the chemotherapeutic agent at the site of the tumor cells. Thus, the payload (i.e. chemotherapy drug) can be deposited directly at the target site rather than being administered in a systemic manner, thus reducing the off-target toxicities associated with chemotherapy.

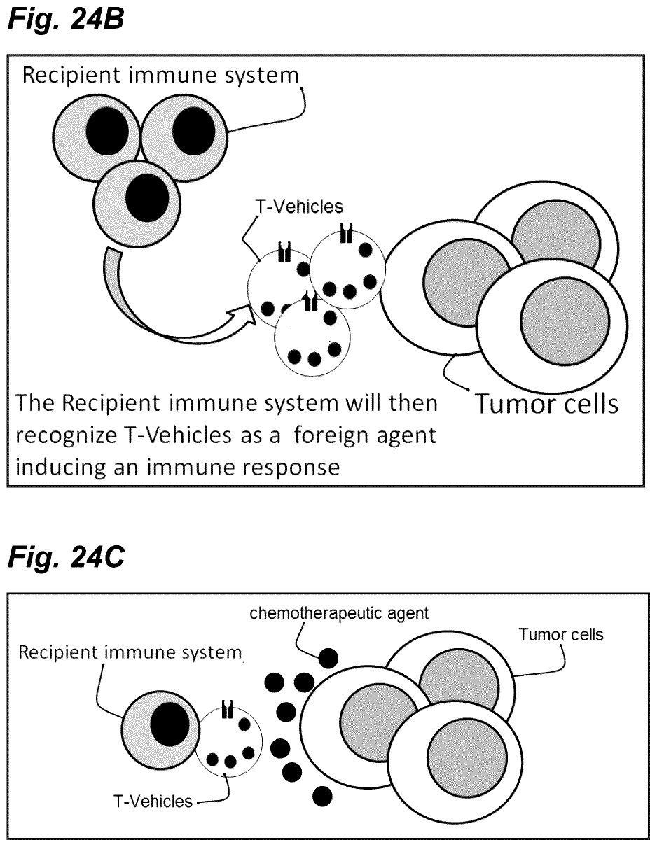

This process is as depicted in FIG. 24A, FIG. 24B, and FIG. 24C. As shown in FIG. 24A, T-Vehicles loaded with chemotherapeutic agent migrate towards the site of inflammation (i.e. tumor cells) and due to the HLA mismatch between T-Vehicles and the Recipient cells, the native antigen receptors of the T-Vehicles does not recognize the Recipient cells, arriving at the Tumor cells without initiating GVHD. As shown in FIG. 24B, the Recipient immune system has targeted the T-Vehicles, which are located at the site of the Tumor cells. As shown in FIG. 24C, under attack by the Recipient immune system, T-Vehicles have released the chemotherapeutic agent at the site of the Tumor cells, thereby avoiding the off target toxicities inherent to state-of-the-art methods of delivering chemotherapy.

T-Vehicles altered with the therapeutic attribute of antimicrobial agents: An antimicrobial is a substance that kills or inhibits the growth of microorganisms such as bacteria, fungi, or protozoans. These agents are typically administered systemically and can be delivered in a more targeted manner if loaded onto T-Vehicles which have the ability to home to sites of inflammation in order to deliver their payload.

T-Vehicles altered with the therapeutic attribute of producing recombinant proteins administered as an adjuvant with immunotherapies: As well as being loaded with exogenous recombinant protein, T-Vehicles can also be engineered using viral (e.g. adenovirus, retrovirus, lentivirus) or non-viral transfection approaches to transgenically express recombinant proteins including cytokines, chemokines, enzymes, tumor antigens and cytokine receptors which can also be designed to act as an adjuvant to other immunotherapeutic interventions in order to enhance T cell persistence, promote expansion, induce homing, etc.

T-Vehicles altered with the therapeutic attribute of expressing transgenic molecules that confer the cells with tumor specificity: In the same way T-Vehicles can be modified with recombinant protein such as cytokines, T-Vehicles can also be engineered using viral (e.g. adenovirus, retrovirus, lentivirus) or non-viral transfection approaches to transgenically express chimeric T cell receptors (CARs).

T-Vehicles altered with the therapeutic attribute of being loaded or engineered with recombinant proteins for the treatment of autoimmune diseases: Autoimmune diseases arise from an inappropriate immune response of the body against substances and tissues normally present in the body. In other words, the immune system mistakes some part of the body as a pathogen and attacks its own cells. This may be restricted to certain organs. The administration of T-Vehicles loaded with recombinant proteins such as IL10, TGFB, IL13 cytokines which will suppress the inflammation can overcome such autoimmune effect by delivering these recombinant proteins directly at the site of inflammation, thus delivering the payload directly where required rather than dispensing the recombinant protein indiscriminately.

T-Vehicles can be engineered to express suicide genes: To allow the rapid and complete elimination of infused cells, T-Vehicles can be incorporated with a safety switches or suicide genes, which can be triggered should toxicity occur. The best validated of the suicide genes is thymidine kinase from herpes simplex virus I (HSV-tk). This enzyme phosphorylates the nontoxic prodrug ganciclovir, which then becomes phosphorylated by endogenous kinases to GCV-triphosphate, causing chain termination and single-strand breaks upon incorporation into DNA, thereby killing dividing cells. Several phase I-II studies have shown that Ganciclovir administration can safely eliminate transferred HSV-tk-modified cells in vivo. More recently, inducible Fas, Fas-associated death domain-containing protein (FADD), and Caspase9 have been considered as alternative non-immunogenic suicide genes. Each of these molecules can act as a suicide switch when fused with an FK-binding protein (FKBP) variant that binds a chemical inducer of dimerization (CID), AP1903, a synthetic drug that has proven safe in healthy volunteers. Administration of this small molecule results in cross-linking and activation of the proapoptotic target molecules. Up to 90% of T cells transduced with inducible Fas or FADD undergo apoptosis after exposure to CID. While promising, elimination of 90% of transduced cells may be insufficient to ensure safety of genetically modified cells in vivo Transgenic expression of the CD20 moleucle, which is normally expressed on B cells, has also been postulated as suicide gene for T cell therapies. This strategy relies on the clinical availability of a humanized anti-CD20 antibody (Rituximab) which is widely used to eliminate both normal and neoplastic B cells expressing the CD20 antigen. Thus, infusion of T cells transgenically expressing human CD20 and subsequent in vivo administration of Rituximab should efficiently eliminate the infused T cell population, although it will also eliminate normal B cells. Thus, T-Vehicles could be modified to express one or a combination of these different suicide genes to control the elimination and the delivery of the payload.

T-Vehicles altered with the therapeutic attribute of loaded and/or engineered to in-vivo imaging: Positron emission tomography (PET) is a nuclear medicine imaging technique that produces a three-dimensional image or picture of functional processes in the body. The system detects pairs of gamma rays emitted indirectly by a positron-emitting radionuclide (tracer), which is introduced into the body on a biologically active molecule. Three-dimensional images of tracer concentration within the body are then constructed by computer analysis. Due to the ability of the T-Vehicle to migrate to the tumor site, T-vehicles can be loaded with radioisotopes to allow the in-vivo detection and determine the location of a tumor site.

Similarly, Iodine-123 (123I or I-123) is a radioactive isotope of iodine used in nuclear medicine imaging, including single photon emission computed tomography (SPECT). This is the most suitable isotope for the diagnostic study of thyroid diseases. The half-life of approximately 13.3 h (hours) is ideal for the 24-h (hour) iodine uptake test and 123I has other advantages for diagnostically imaging thyroid tissue and thyroid cancer metastasis. Iodine can be used in a safe manner to image, or treat the thyroid tumor, due to the selective capture of Iodine in the “Iodine trap” by the hydrogen peroxide generated by the enzyme thyroid peroxidase (TPO). In this way, T-vehicles could be modified with Thyroid peroxidase or thyroperoxidase (TPO) to trap Iodine which can then be used to image/or kill the T-Vehicles.

Skilled artisans are encouraged to recognize that the therapeutic attribute for any given therapeutic purpose of the T-Vehicles can be created by many techniques including but not limited to any of the following:
a) genetic modification with a viral vector such as retrovirus, adenovirus, Adeno-associated virus or lentivirus, and/or
b) genetic modification by non-viral vectors including the use of DNA and/or RNA vectors which are incorporated by physical and/or chemical techniques such as electroporation and/or lipofection methods using transposons and transposases (e.g. Sleeping Beauty), and/or Piggybac techniques, and/or
c) genetic modification for the inclusion of one or more transgenes that modify T-Vehicle migration, incorporate a suicide gene, improve recipient immune reconstitution (e.g. cytokine production), and/or elicit a direct anti-viral or anti-tumor effect (e.g. chimeric antigen receptor) or suppress the immune response for the treatment of auto immune diseases, and/or
d) genetic modification to improve the migration of the T-Vehicle by the expression of one or more chemokine receptors such as CCR1, CCR2, CCR3, CCR4, CCR5, CCR6, CCR7, CCR8, CCR9, CCR10, CXCR1, CXCR2, CXCR3-A, CXCR5, CXCR6, CX₃CR1, and/or XCR1 to improve the migration of the T-Vehicle, and/or
e) genetic modification to improve recipient immune reconstitution by T-Vehicle expression of one or more cytokines such as GM-CSF, TNFa, INFy, IL2, IL8 IL15, IL7, IL12, IL21 or IL26 or through the expression or over expression of co-stimulatory molecules CD80,CD86, 41BBL, OX40L, and/or
f) genetic modification to induce T-Vehicle death by the expression of one or more suicide genes such as thymidine kinase TK gene, CD20, CD19 or iCaspase9, and/or
g) genetic modification to elicit direct anti-viral or anti-tumor effects including but not limited to the expression of one or more transgenes such as chimeric antigen receptors (CARs) that recognize tumor targets through single-chain variable fragments (scFv) isolated from specific antibodies linked with i) an extracellular spacer such as by the use of the CH2CH3 sequence derived from the IgG-FC region, or ii) a trans-membrane component including but not limited to the sequence of CD28, CD4, CD3 or CD8, iii) CD3ζ endodomain or iv) by the expression of natural ligands such as cytokines or cytokines receptors encoding the CD3ζ endodomain, and/or
h) genetic modification to suppress the immune response for the treatment of auto immune diseases for example by the expression of transgenes that produce one or more immunosuppressive cytokines such as IL4, IL6, IL10, IL13, TFGβ, or by the expression of competitor ligands such as CTLA-4, PD1.

Skilled artisans are encouraged to recognize that the therapeutic purpose of the T-Vehicles can be wide ranging including but not limited to any of the following:
a) as a biological vehicle to carry DNA, RNA, recombinant proteins, peptides or aptamers
b) as a biological vehicle to carry chemical compound, and/or
c) as a biological vehicle allows to carry chemical compound with therapeutic purpose including but not limited to chemotherapy drugs, small molecules, nanoparticles, hormonal agonist or antagonist, anti-viral, anti-fungal, anti-parasitic agent, and/or
d) as a biological vehicle to carry chemical compound(s) with no therapeutic purpose but secondary gain including but not limited to in-vivo identification and imaging that will allow to identify metastatic disease sites.

Each of the applications, patents, and papers cited in this application and as well as in each document or reference cited in each of the applications, patents, and papers (including during the prosecution of each issued patent; “application cited documents”), pending U.S. Publication Nos. 2005/0106717 A1 and 2008/0227176 A1, and each of the PCT and foreign applications or patents corresponding to and/or claiming priority from any of these applications and patents, and each of the documents cited or referenced in each of the application cited documents, are hereby expressly incorporated herein.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

Those skilled in the art will recognize that numerous modifications can be made to this disclosure without departing from the spirit of the inventions described herein. Therefore, it is not intended to limit the breadth of the invention to embodiments and examples described. Rather, the scope of the invention is to be interpreted by the appended claims and their equivalents.

What is claimed is:

1. A composition of therapeutic cells for infusion into recipients, the composition comprising a population of human T-cells including a population of T-Vehicles, wherein the T-vehicles comprise a population of antigen specific human T cells which have a native T cell receptor that recognizes a non-human antigen and the T-Vehicles being genetically modified with at least one therapeutic attribute;
wherein when the composition of therapeutic cells is administered to a recipient, the T-Vehicles migrate to a site of inflammation.

2. The T-Vehicles of claim 1 wherein said therapeutic attribute is a chimeric antigen receptor.

3. The composition of therapeutic cells of claim 1, wherein said population of human T cells are obtained by seeding PBMCs and medium into a device that includes a growth surface comprised of gas permeable material at a medium volume to growth surface area ratio of at least 2 mL/cm² and stimulating the PBMCs with the non-human antigen to activate the growth of T cells that have native antigen specificity to the non-human antigen.

4. The composition of cells of claim 1, wherein when the composition of therapeutic cells is administered to a recipient, the site of inflammation is a tumor.

5. The composition of therapeutic cells of claim 1, wherein said non-human antigen is a viral antigen.

6. The T-Vehicles of claim 1, wherein said human T cells being further genetically modified to express a receptor that recognizes a cytokine.

7. The T-Vehicles cells of claim 6, wherein said cytokine is IL4.

8. The T-Vehicles of claim 1 wherein said T-Vehicles being further genetically modified to include a suicide gene, wherein the suicide gene is caspase 9.

9. The T-Vehicles of claim 5, wherein said native T cell receptor only recognizes a single epitope of said viral antigen.

10. The T-Vehicles of claim 2, wherein, the chimeric antigen receptor recognizes a tumor target.

11. The T-Vehicles of claim 1, wherein the T-Vehicles have been genetically modified by viral transfection.

12. The composition of therapeutic cells of claim 1, wherein the composition of therapeutic cells are administered to a recipient independent of the HLA of the recipient.

13. The composition of therapeutic cells of claim 1, wherein the composition of therapeutic cells are administered to a recipient that is a HLA mismatch to the composition of therapeutic cells.

14. The T-Vehicles of claim 1 wherein said therapeutic attribute is the expression of IL-2.

15. The T-Vehicles of claim 1 wherein said therapeutic attribute is the expression of IL7.

16. The T-Vehicles of claim 1 wherein said therapeutic attribute is the expression of IL15.

17. The composition of therapeutic cells of claim 1 wherein the cells are frozen and stored in an allogenic cell bank.
